(19) United States
(12) Patent Application Publication
(10) Pub. No.: US 2024/0003901 A1
(43) Pub. Date: Jan. 4, 2024

(54) ASSAYS FOR FABRY DISEASE TREATMENT

(71) Applicant: Sangamo Therapeutics, Inc., Brisbane, CA (US)

(72) Inventor: Joshua Nelson HOLTER, Brisbane, CA (US)

(73) Assignee: Sangamo Therapeutics, Inc., Brisbane, CA (US)

(21) Appl. No.: 18/247,384

(22) PCT Filed: Sep. 30, 2021
(86) PCT No.: PCT/US2021/052982

§ 371 Date: Mar. 30, 2023

Related U.S. Application Data
(60) Provisional application No. 63/086,523, filed on Oct. 1, 2020.

(51) Int. Cl. G01N 33/68; A61K 48/00; A61K 31/445; A61K 31/439; C12N 15/86; A61K 45/06; A61K 31/517

(52) U.S. Cl. CPC G01N 33/6854; A61K 48/005; A61K 31/445; A61K 31/439; C12N 15/86; A61K 45/06; A61K 31/517; C12Y 302/01022; G01N 2800/28

(57) ABSTRACT

The disclosure provides the enzyme and non-enzyme replacement therapies for treating Fabry disease in a human subject, determined based on measuring the presence or absence of an anti-α-Galactosidase A (GLA) neutralizing antibody in a biological sample of the subject, as measured by an anti-GLA neutralizing antibody assay.

### CROSS REFERENCE TO RELATED APPLICATIONS

This application claims the benefit of the filing date of U.S. Provisional Patent Application No. 63/086,523, filed Oct. 1, 2020, the contents of which are incorporated herein by reference in their entirety.

### FIELD OF DISCLOSURE

The present disclosure provides the enzyme and non-enzyme replacement therapies for treating Fabry disease in a human subject, determined based on measuring the presence or absence of an anti-α-Galactosidase A (GLA) neutralizing antibody in a biological sample of the subject, as measured by an anti-GLA neutralizing antibody assay.

### BACKGROUND OF THE DISCLOSURE

Fabry disease is an X-linked lysosomal storage disorder resulting from a deficiency of the enzyme α-Galactosidase A (GLA). The resulting failure to hydrolyze the terminal α-galactosyl moiety from globotriaosylceramide (Gb3) causes accumulation of Gb3 in lysosomes and elsewhere in the cell. Early characteristic clinical manifestations include severe neuropathic pain (acroparesthesia), skin lesions (angiokeratomas), and ocular signs (cornea verticillata). Later in life, cardiac, renal, and cerebrovascular complications are responsible for severe morbidity and a shortened lifespan. Desnick et al., α-Galactosidase A Deficiency: Fabry Disease, in: Beaudet et al. (Ed.), The Online Metabolic and Molecular Bases of Inherited Disease, The McGraw-Hill Companies, Inc., New York, NY (2014); Van der Veen SJ et al., Mol Genet Metab. 126(2):162-168 (2019).

Since 2001, patients with Fabry disease have been treated with two different enzyme replacement therapies (ERTs), based on infusion of recombinant enzymes (agalsidase-α and agalsidase-(3). Eng et al., N Engl J Med 345: 9-16 (2001); Schiffmann et al., JAMA 285: 2743-2749 (2001); Lenders et al., J Am Soc Nephroi. 27(1):256-64 (2016). Treatment with ERT results in a notable reduction of Gb3 and its deacylated form lysoglobotriaosylsphingosine (lysoGb3) in plasma and urine (Arends et al., Mol Genet Metab 121(2):157-161 (2017); Rombach et al., PLoS One 7(10): e47805 (2012)), and morphological clearance of storage material in endothelial cells and, to a lesser extent, podocytes (Tondel et al., J Am Soc Nephrol 24 (1): 137-148 (2013); Thurberg et al., Circulation 119(19): 2561-2567 (2009)).

Studies suggested that infusion of recombinant enzyme can lead to formation of the anti-GLA neutralizing antibodies, resulting in short-term acute complications, as well as deleterious long-term effects by therapy inhibition, resulting in severely decreased Gb3 and lyso-Gb3 depletion. Lenders et al., J Am Soc Nephrol. 27(1):256-64 (2016). In male patients with classical Fabry disease, treatment with ERT delays the occurrence of complications, especially when treatment is initiated before the onset of irreversible organ damage. However, more than a half of classically affected male patients treated with ERT develop anti-GLA neutralizing antibodies. In female patients and patients with a non-classical disease phenotype, antibody formation against the administered recombinant enzyme is rarely observed. Van der Veen SJ et al., Mol Genet Metab. 126(2):162-168 (2019).

Accordingly, there is a need for targeted therapeutic strategies that identify patients who are more likely to respond to a particular Fabry disease therapy and, thus, improve the clinical outcome for patients diagnosed with Fabry disease.

### SUMMARY OF THE DISCLOSURE

In some aspects, the disclosure is directed to a method of treating Fabry disease in a human subject in need thereof comprising administering a therapy for Fabry disease to the subject, wherein the subject is identified as an anti-α-Galactosidase A (GLA) neutralizing antibody negative subject when a biological sample of the subject is analyzed, wherein the anti-GLA neutralizing antibody negative subject has a biological sample having lower than about 30% inhibition of α-Galactosidase A activity as measured by an anti-GLA neutralizing antibody assay.

In some aspects, the disclosure is directed to a method of identifying a human subject suitable for a therapy for Fabry disease comprising measuring the presence of an anti-GLA neutralizing antibody in a biological sample of the subject, wherein the subject suitable for a therapy is an anti-GLA neutralizing antibody negative subject when a biological sample of the subject is analyzed, wherein the anti-GLA neutralizing antibody negative subject has a biological sample having lower than about 30% inhibition of α-Galactosidase A activity as measured by an anti-GLA neutralizing antibody assay.

In some aspects, the disclosure is directed to a method of treating Fabry disease in a human subject in need thereof comprising administering a therapy for Fabry disease to the subject, wherein the subject is identified as an anti-α-Galactosidase A (GLA) neutralizing antibody negative subject when a biological sample of the subject is analyzed, wherein biological sample is a serum sample, which is diluted at minimum required dilution (MRD)15 or lower.

In some aspects, the disclosure is directed to a method of identifying a human subject suitable for a therapy for Fabry disease comprising measuring the presence of an anti-GLA neutralizing antibody in a biological sample of the subject, wherein the subject suitable for a therapy is an anti-GLA neutralizing antibody negative subject when a biological sample of the subject is analyzed, wherein biological sample is a serum sample, which is diluted at MRD15 or lower.

In some aspects, the serum sample is mixed with α-Galactosidase A (GLA).

In some aspects, the GLA is at a concentration of less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about 20 ng/ml, or less than about 10 ng/ml. In some aspects, the GLA is at a concentration of about 20 ng/ml.

In some aspects, the serum sample and the GLA mixture are incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

In some aspects, the serum sample and the GLA mixture are incubated for a duration between about 1 and about 15 hours, between about 2 and about 14 hours, between about 3 and about 13 hours, between about 4 and about 12 hours, between about 5 and about 11 hours, between about 6 and about 10 hours, or between about 7 and about 9 hours.

In some aspects, the serum sample is mixed with a reaction mix. In some aspects, the reaction mix comprises a substrate and an inhibitor. In some aspects, the substrate comprises 4-methylumbelliferyl (4-MU)-α-D-galactopyranoside. In some aspects, the substrate comprises 4-Nitrophenyl α-D-galactopyranoside. In some aspects, the inhibitor comprises N-Acetylgalactosamine (GALNAc).

In some aspects, the substrate is at a concentration of at least about 1.1 mM, at least about 1.2 mM, at least about 1.3 mM, at least about 1.4 mM, at least about 1.5 mM, at least about 1.6 mM, at least about 1.7 mM, at least about 1.8 mM, at least about 1.9 mM, at least about 2 mM, at least about 2.1 mM, at least about 2.2 mM, at least about 2.3 mM, at least about 2.4 mM, at least about 2.5 mM, at least about 2.6 mM, at least about 2.7 mM, at least about 2.8 mM, at least about 2.9 mM, at least about 3 mM, at least about 3.1 mM, at least about 3.2 mM, at least about 3.3 mM, at least about 3.4 mM, at least about 3.5 mM, at least about 3.6 mM, at least about 3.7 mM, at least about 3.8 mM, at least about 3.9 mM. at least about 4 mM, at least about 4.1 mM, at least about 4.2 mM, at least about 4.3 mM, at least about 4.4 mM, at least about 4.5 mM, at least about 4.6 mM, at least about 4.7 mM, at least about 4.8 mM, at least about 4.9 mM, or at least about 5 mM.

In some aspects, the inhibitor is at a concentration of less than about 200 mM, less than about 195 mM, less than about 190 mM, less than about 185 mM, less than about 180 mM, less than about 175 mM, less than about 170 mM, less than about 165 mM, less than about 160 mM, less than about 155 mM, less than about 150 mM, less than about 145 mM, less than about 140 mM, less than about 135 mM, less than about 130 mM, less than about 125 mM, less than about 120 mM, less than about 115 mM, or less than about 110 mM.

In some aspects, the reaction mix and the serum sample are mixed in a high throughput plate. In some aspects, the reaction mix and the serum sample mixture are incubated at room temperature at revolutions per minute (RPM) 300, 400, 500, or 600.

In some aspects, the method disclosed herein further comprising adding a stop buffer to the mixture after incubation. In some aspects, the incubation period is at least about 30 minutes, at least about 35 minutes, at least about 40 minutes, at least about 45 minutes, at least about 50 minutes, at least about 55 minutes, at least about 60 minutes, at least about 65 minutes, at least about 70 minutes, at least about 75 minutes, or at least about 80 minutes.

In some aspects, the stop buffer comprises glycine. In some aspects, the stop buffer is at a volume of less than about 1 mL, less than about 900 uL, less than about 800 uL, less than about 700 uL, less than about 600 uL, less than about 500 uL, less than about 400 uL, less than about 300 uL, less than about 200 uL, or less than about 100 uL.

In some aspects, the anti-GLA neutralizing antibody negative subject has a biological sample having lower than about 30% inhibition of α-Galactosidase A activity as measured by an anti-GLA neutralizing antibody assay.

In some aspects, the method further comprises administering Fabry disease therapy.

In some aspects, the sample has lower than about 15%, about 16%, about 17% about 18%, about 19%, about 20%, about 21%, about 22%, about 23%, about 24%, about 25%, about 26%, about 27%, about 28%, about 29%, or about 30% inhibition of α-Galactosidase A activity.

In some aspects, the sample has lower than about 27% inhibition of α-Galactosidase A activity. In some aspects, the sample has lower than about 20% inhibition of α-Galactosidase A activity. In some aspects, the sample has lower than about 15% inhibition of α-Galactosidase A activity. In some aspects, the sample has lower than about 10% inhibition of α-Galactosidase A activity. In some aspects, the sample has lower than 1% inhibition of α-Galactosidase A activity. In some aspects, the sample has no inhibition of α-Galactosidase A activity.

In some aspects, the sample has about 10% to about 20% inhibition of α-Galactosidase A activity. In some aspects, the sample has about 20% to about 30% inhibition of α-Galactosidase A activity.

In some aspects, the subject has been administered with an enzyme replacement therapy for Fabry disease prior to the administering and/or the measuring (“pre-treatment”). In some aspects, the therapy for Fabry disease is an enzyme replacement therapy. In some aspects, the enzyme replacement therapy comprises a recombinant α-Galactosidase A (GLA) protein or a gene expressing GAL.

In some aspects, the enzyme replacement therapy comprises administering galafold, ST-920, AVR-RD-01, FLT-190, or any combination thereof.

In some aspects, the enzyme replacement therapy comprises a recombinant α-Galactosidase A (GLA) protein in combination with an active site-specific chaperone (ASSC) for the GLA. In some aspects, the ASSC is 1-deoxygalactonojirimycin.

In some aspects, the enzyme replacement therapy comprises agalsidase alpha and/or beta or a gene expressing agalsidase alpha and/or beta. In some aspects, the enzyme replacement therapy comprises fabrazyme, Replagal, PRX-102, or any combination thereof.

In some aspects, the enzyme replacement therapy comprises a gene therapy. In some aspects, the gene therapy comprises a vector encoding the enzyme. In some aspects, the gene therapy comprises administering ST-920, AVR-RD-01, FLT-190, or any combination thereof. In some aspects, the gene therapy is delivered by a lipid nanoparticle.

In some aspects, the vector comprises an mRNA encoding a human GLA protein or agalsidase alpha and/or beta. In some aspects, the vector is a viral vector. In some aspects, the viral vector comprises an adeno-associated virus (AAV) vector or a lentiviral vector.

In some aspects, the therapy for Fabry disease comprises a non-enzyme replacement therapy. In some aspects, the therapy for Fabry disease comprises lucerastat, venglustat, apabetalone, or any combination thereof.

In some aspects, the pre-treatment is an enzyme replacement therapy. In some aspects, the pre-treatment comprises a recombinant α-Galactosidase A (GLA) protein or a gene expressing GAL.

In some aspects, the enzyme replacement therapy for the pre-treatment comprises administering galafold, ST-920, AVR-RD-01, FLT-190, or any combination thereof.

In some aspects, the enzyme replacement therapy for the pre-treatment comprises agalsidase alpha and/or beta or a gene expressing agalsidase alpha and/or beta.

In some aspects, the enzyme replacement therapy for the pre-treatment comprises fabrazyme, Replagal, PRX-102, or any combination thereof.

In some aspects, the enzyme replacement therapy for the pre-treatment comprises a gene therapy. In some aspects, the gene therapy comprises a vector encoding the enzyme. In some aspects, the gene therapy comprises administering ST-920, AVR-RD-01, FLT-190, or any combination thereof. In some aspects, the vector comprises an mRNA encoding a human GLA protein or agalsidase alpha and/or beta. In some aspects, the vector is a viral vector. In some aspects, the viral vector comprises an adeno-associated virus (AAV) vector or a lentiviral vector. In some aspects, the gene therapy is delivered by a lipid nanoparticle.

In some aspects, Fabry disease is type 1 classic phenotype or type 2 later-onset phenotype.

In some aspects, the biological sample is serum.

In some aspects, the anti-GLA neutralizing antibody assay is standardized by an antibody designated as RP-01. In some aspects, the assay standardization comprises determining a GLA drug diluent concentration by measuring the effect of the GLA drug level on the inhibitory effect of the RP-01 antibody.

In some aspects, the disclosure is directed to a method of standardizing an anti-GLA neutralizing antibody assay which comprises determining a GLA drug diluent concentration by measuring the effect of the GLA drug level on the inhibitory effect of an antibody designated as RP-01. In some aspects, the RP-01 is a polyclonal antibody.

In some aspects, the standardization comprises determining a GLA drug diluent concentration by measuring the effect of the GLA drug level on the inhibitory effect of the RP-01 antibody. In some aspects, the GLA drug diluent concentration is about 200 ng/ml, about 150 ng/ml, about 100 ng/ml, about 90 ng/ml, about 80 ng/ml, about 70 ng/ml, about ng/ml, about 50 ng/ml, about 40 ng/ml, about 30 ng/ml, about 20 ng/ml, or about 10 ng/ml. In some aspects, the GLA drug diluent concentration is about 40 ng/ml. In some aspects, the GLA drug diluent concentration is about 20 ng/ml.

In some aspects, the inhibitory effect of the RP-01 antibody is represented as % inhibition of α-Galactosidase A activity as measured by the anti-GLA neutralizing antibody assay using different concentrations of the RP-01 antibody. In some aspects, the inhibitory effect of RP-01 is represented as about 20% inhibition of α-Galactosidase A activity at about 50 ug/ml RP-01 concentration. In some aspects, the inhibitory effect of RP-01 is represented as about 30% inhibition of α-Galactosidase A activity at about 100 ug/ml RP-01 concentration. In some aspects, the inhibitory effect of RP-01 is represented as about 40% inhibition of α-Galactosidase A activity at about 150 ug/ml RP-01 concentration.

In some aspects, the GLA drug has a minimal required dilution (MRD) of about 1 fold, about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, about 41 fold, about 42 fold, about 43 fold, about 44 fold, about 45 fold, about 46 fold, about 47 fold, about 48 fold, about 49 fold, about 50 fold, about 51 fold, about 52 fold, about 53 fold, about 54 fold, about 55 fold, about 56 fold, about 57 fold, about 58 fold, about 59 fold, about 60 fold, about 61 fold, about 62 fold, about 63 fold, about 64 fold, about 65 fold, about 66 fold, about 67 fold, about 68 fold, about 69 fold, about 70 fold, about 71 fold, about 72 fold, about 73 fold, about 74 fold, about 75 fold, about 76 fold, about 77 fold, about 78 fold, about 79 fold, about 80 fold, about 81 fold, about 82 fold, about 83 fold, about 84 fold, about 85 fold, about 86 fold, about 87 fold, about 88 fold, about 89 fold, about 90 fold, about 91 fold, about 92 fold, about 93 fold, about 94 fold, about 95 fold, about 96 fold, about 97 fold, about 98 fold, about 99 fold, or about 100 fold.

In some aspects, the disclosure is directed to a kit comprising the anti-GLA neutralizing antibody assay as described herein, wherein the kit comprises: (a) an assay buffer, (b) a substrate, (c) GALNAc inhibitor, (d) stop solution, and (e) an insert comprising instructions for use of the kit.

In some aspects, the disclosure is directed to a method of treating Fabry disease in a human subject in need thereof comprising administering a non-enzyme replacement therapy for Fabry disease to the subject, wherein the subject is identified as an anti-α-Galactosidase A (GLA) neutralizing antibody positive subject when a biological sample of the subject is analyzed, wherein the anti-GLA neutralizing antibody positive subject has a biological sample having higher than about 10% inhibition of α-Galactosidase A activity as measured by an anti-GLA neutralizing antibody assay.

In some aspects, the disclosure is directed to a method of identifying a human subject suitable for a non-enzyme replacement therapy for Fabry disease comprising measuring the presence of an anti-GLA neutralizing antibody in a biological sample of the subject, wherein the subject suitable for a therapy is an anti-GLA neutralizing antibody positive subject when a biological sample of the subject is analyzed, wherein the anti-GLA neutralizing antibody positive subject has a biological sample having higher than about 10% inhibition of α-Galactosidase A activity as measured by an anti-GLA neutralizing antibody assay. In some aspects, the method further comprises administering a therapy that is not an enzyme replacement therapy.

In some aspects, the disclosure is directed to a method of identifying a human subject who is not eligible for an enzyme replacement therapy for Fabry disease comprising measuring the presence of an anti-GLA neutralizing antibody in a biological sample of the subject, wherein the sample having higher than about 10% inhibition of α-Galactosidase A activity as measured by the anti-GLA neutralizing antibody assay is identified as the anti-GLA neutralizing antibody positive sample, and wherein the subject not eligible for the enzyme replacement therapy for Fabry disease has the anti-GLA neutralizing antibody positive sample. In some aspects, the method further comprises administering Fabry disease therapy that is not an enzyme replacement therapy. In some aspects, the therapy comprises administering lucerastat, venglustat, or apabetalone.

### BRIEF DESCRIPTION OF THE DRAWINGS

FIG. 1 shows human α-Galactosidase A (GLA) neutralizing antibody assay format.

FIG. 2 shows performance of the GLA neutralizing antibody assay across 6 different runs with 50 different serum donors. (• represents sample results; ⋆ represents statistical outlier; - - - represents median; and—represents statistical outlier limits).

FIG. 3 shows antibody screen for the GLA neutralizing antibody. The GLA neutralizing performance (percent (%) inhibition of α-Galactosidase A activity) was measured for 40 different antibodies.

FIG. 4 shows the GLA neutralizing (positive control) antibody performance; e.g., the enzymatic neutralizing ability (percent (%) inhibition of α-Galactosidase A activity) of affinity purified RP-01 antibody, the flow through RP-01, and RP-01 antibody at different concentrations (ug/ml).

FIGS. 5A-5G show the GLA neutralizing antibody assay optimization for sensitivity and drug tolerance (DT). FIG. 5A shows the effect of buffer enzyme concentrations (5 ng/ml GLA, 50 ng/ml GLA, 100 ng/ml, and 200 ng/ml) on the enzymatic neutralizing ability (% inhibition of α-Galactosidase A activity) of polyclonal antibody RP-01 at different antibody concentrations (ug/ml). FIG. 5B shows the ability to detect RP-01 neutralizing antibody in the presence of supraphysiological levels of circulating GLA ([100 ug RP-01 (100 ng/ml GLA drug level (DL)]; [150 ug RP-01 (100 ng/ml DL]; [100 ug RP-01 (50 ng/ml DL]; and [150 ug RP-01 (50 ng/ml DL]). FIG. 5C shows the effect of the initial dilution of human sera at 1:10 (minimum required dilution (MRD10)) on the enzymatic neutralizing ability (% inhibition of α-Galactosidase A activity) of RP-01 antibody at different concentrations (0 ug, 50 ug, 100 ug, and 150 ug). FIG. 5D shows the effect of the initial dilution of human sera at 1:20 (minimum required dilution (MRD20)) on the enzymatic neutralizing ability (% inhibition of α-Galactosidase A activity) of RP-01 antibody at different concentrations (0 ug, 50 ug, 100 ug, and 150 ug). FIG. 5E shows the effect of the two hour incubation time of the serum and GLA enzyme buffer on the enzymatic neutralizing ability (% inhibition of α-Galactosidase A activity) of RP-01 antibody at different concentrations (0 ug, 50 ug, 100 ug, and 150 ug). FIG. 5F shows the effect of the overnight incubation of the serum and GLA enzyme buffer on the enzymatic neutralizing ability (% inhibition of α-Galactosidase A activity) of RP-01 antibody at different concentrations (0 ug, 50 ug, 100 ug, and 150 ug). FIG. 5G shows the effect of heat pre-treatment of the MRD10 sample on the enzymatic neutralizing ability (% inhibition of α-Galactosidase A activity) of RP-01 antibody at different concentrations (0 ug, 50 ug, 100 ug, and 150 ug).

### DETAILED DESCRIPTION OF THE DISCLOSURE

The present disclosure provides the enzyme and non-enzyme replacement therapies for treating Fabry disease in a human subject determined based on measuring the presence or absence of an anti-α-Galactosidase A (GLA) neutralizing antibody in a biological sample of the subject as measured by an anti-GLA neutralizing antibody assay.

### I. Terms

In order that the present disclosure can be more readily understood, some terms are first defined. As used in this application, except as otherwise expressly provided herein, each of the following terms shall have the meaning set forth below. Additional definitions are set forth throughout the application.

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

The terms “α-Galactosidase A,” “α-Gal A,” and “GAL” are used interchangeably and refer to a protein with enzymatic activity comprising hydrolysis of terminal, non-reducing α-D-galactose residues in α-D-galactosides, including galactose oligosaccharides, galactomannans and galactolipids. In some aspects, α-Gal A comprises the enzyme described by IUBMB Enzyme Nomenclature EC 3.2.1.22 (as described, for example, in Suzuki et al., J. Biol. Chem. 245:781-786(1970); Wiederschain, G. and Beyer, E. Dokl. Akad. Nauk S.S.S.R. 231:486-488 (1976)). In some aspects, α-Gal A comprises a protein encoded by a nucleic acid comprising the human GLA gene, for example, the human α-Gal A gene defined by GenBank Accession No. NM 000169. In some aspects, α-Gal A comprises a protein comprising the amino acid sequence defined by GenBank Accession No. NP 000160.

In some aspects, GAL can be obtained from a cell endogenously expressing the α-Gal A, or the α-Gal A can be a recombinant human α-Gal A (rha-Gal A), as described herein. In some aspects, the rha-Gal A is a full length wild-type α-Gal A. In some aspects, the rha-Gal A comprises a subset of the amino acid residues present in a wild-type α-Gal A, wherein the subset includes the amino acid residues of the wild-type α-Gal A that form the active site for substrate binding and/or substrate reduction. In some aspects, an rha-Gal A that is a fusion protein comprising the wild-type α-Gal A active site for substrate binding and/or substrate reduction, as well as other amino acid residues that can or may not be present in the wild type α-Gal A.

α-Gal A can be obtained from commercial sources or can be obtained by synthesis techniques known to a person of ordinary skill in the art. The wild-type enzyme can be purified from a recombinant cellular expression system (e.g., mammalian cells such as CHO cells, or insect cells, see e.g., U.S. Pat. Nos. 5,580,757; 6,395,884; 6,458,574; 6,461,609; 6,210,666; 6,083,725), human placenta, or animal milk.

Other synthesis techniques for obtaining α-Gal A suitable for pharmaceutical use can be found, for example, in U.S. Pat. Nos. 7,560,424; 7,396,811; 423,135; 6,534,300; and 6,537,785; U.S. Published Application Nos. 2009/0203575; 2009/0029467; 2008/0299640; 2008/0241118; 2006/0121018; 2005/0244400; 2007/0280925; and 2004/0029779, and International Published Application No. 2005/077093.

In some aspects, the α-Gal A is agalsidase alpha, produced by genetic engineering technology in a human cell line. Agalsidase alpha is available as Replagal®, from Shire Plc. (Dublin, Ireland). In some aspects, the α-Gal A is agalsidase beta, produced by recombinant DNA technology in a Chinese hamster ovary (CHO) cell line. Agalsidase beta is available as Fabrazyme®, from Sanofi Genzyme (Cambridge, Mass.). In some aspects, the α-Gal A is a recombinant human α-Gal A produced in CHO cells transformed with an expression vector encoding the human α-Gal A gene (JCR Pharmaceuticals Co. Ltd, (Japan)), identified as JR-051.

In addition to proteins that comprise an amino acid sequence that is identical to the human α-Gal A proteins described herein, this disclosure also encompasses α-Gal A proteins that are “substantially similar” thereto. Proteins described herein as being “substantially similar” to a reference protein include proteins that retain some structural and functional features of the native proteins yet differ from the native amino acid sequence at one or more amino acid positions (i.e., by amino acid substitutions).

Proteins altered from the native sequence can be prepared by substituting amino acid residues within a native protein and selecting proteins with the desired activity. For example, amino acid residues of an α-Gal A protein can be systematically substituted with other residues and the substituted proteins can then be tested in standard assays for evaluating the effects of such substitutions on the ability of the protein to hydrolyze a terminal, non-reducing α-D-galactose residues in α-D-galactosides, including galactose oligosaccharides, galactomannans and galactolipids, and/or on the ability to treat or prevent Fabry disease.

In some aspects, to retain functional activity, conservative amino acid substitutions are made. As used herein, “conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an α-Gal A protein is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

In some aspects, an α-Gal A protein of the disclosure is at least about 70%, 71%, 72%, 73%, 74%, 75%, 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, or 99% identical to the amino acid sequence of an α-Gal A protein described herein or known in the art.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (I Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and)(BLAST programs (version 2.0) of Altschul, et al. (1990) J Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the)(BLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

An “antibody” (Ab) includes, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, Cm, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FRs). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term “anti-GAL antibody,” for example, includes a full antibody having two heavy chains and two light chains that specifically binds to α-Gal A and antigen-binding portions of the full antibody.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to α-Gal A is substantially free of antibodies that bind specifically to antigens other than α-Gal A). An isolated antibody that binds specifically to α-Gal A can, however, have cross-reactivity to other antigens, such as α-Gal A molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

The term “polyclonal antibodies” (pAbs) refers to a mixture of heterogeneous antibodies which are usually produced by different B cell clones in the body. They can recognize and bind to many different epitopes of a single antigen. In some aspects, the RP-01 antibody, described herein, is a polyclonal antibody.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one aspect of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-GAL antibody binds specifically to GAL.

The term “anti-GLA neutralizing antibody,” “anti-GLA NAb,” “anti-drug antibody,” “ADA,” “neutralizing anti-drug antibody,” or “neutralizing ADA,” refers to an antibody that binds and inactivates (neutralizes) α-Gal A enzyme. In some aspects, if the anti-GLA neutralizing antibodies are present, the enzyme replacement therapy is directly inactivated (neutralized) by the anti-GLA neutralizing antibodies in the plasma (Linthorst et al., Kidny Int 66:1589-1595 (2004); Lenders et al., J Allergy Clin Immunol 141:2289-2292.e7 (2018)).

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-GLA 3 antibody described herein, include (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. km refers to the association rate constant of, e.g., an antibody to an antigen, and k_{off} refers to the dissociation of, e.g., an antibody to an antigen. The kₒₙ, and k_{off} can be determined by techniques known to one of ordinary skill in the art, such as immunoassays (e.g., enzyme-linked immunosorbent assay (ELISA)), BIACORE®, BLI (Bio-layer interferometry), or kinetic exclusion assay (KINEXA®).

As used herein, the terms “specifically binds,” “specifically recognizes,” “specific binding,” “selective binding,” and “selectively binds,” are analogous terms in the context of antibodies and refer to molecules (e.g., antibodies) that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIACORE®, KINEXA® 3000 instrument (Sapidyne Instruments, Boise, ID), or other assays known in the art. In a specific aspect, molecules that specifically bind to an antigen bind to the antigen with a K_{A} that is at least 2 logs, 2.5 logs, 3 logs, 4 logs or greater than the K_{A} when the molecules bind to another antigen.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 10⁻⁹ M or less, and most preferably between 10⁻⁸ M and 10⁻¹⁰ M or less, when determined by, e.g., immunoassays (e.g., ELISA) surface plasmon resonance (SPR) technology in a BIACORE™ 2000 instrument using the predetermined antigen, or BLI (Bio-layer interferometry) but does not bind with high affinity to unrelated antigens.

The term “nucleic acid molecule,” as used herein, is intended to include DNA molecules and RNA molecules. A nucleic acid molecule can be single-stranded or double-stranded, and can be cDNA.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Some vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, some vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses, adeno-associated viruses (“AAV”), and lentiviruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because some modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The term “Fabry disease” refers to classical Fabry disease, late-onset Fabry disease, and hemizygous females having mutations in the gene encoding an α-Gal A. The term “Fabry disease,” as used herein, further includes any condition in which a subject exhibits lower than normal endogenous α-Gal A activity. Fabry disease is referred to by many other names, for example, alpha-galactosidase A deficiency, Anderson-Fabry disease, angiokeratoma corporis diffusum, angiokeratoma diffuse, ceramide trihexosidase deficiency, Fabry's disease, GLA deficienc, and hereditary dystopic lipidosis. In some aspects, Fabry disease is type 1 classic phenotype or type 2 later-onset phenotype.

The term “enzyme replacement therapy” or “ERT” refers to the introduction of a non-native, purified enzyme into an individual having a deficiency in such enzyme (e.g., α-Gal A). The administered enzyme can be obtained from natural sources or by recombinant expression. The term also refers to the introduction of a purified enzyme in an individual otherwise requiring or benefiting from administration of a purified enzyme, e.g., suffering from protein insufficiency. The introduced enzyme can be a purified, recombinant enzyme produced in vitro, or enzyme purified from isolated tissue or fluid, such as, e.g., placenta or animal milk, or from plants.

The term “co-formulation” refers to a composition comprising an enzyme, such as an enzyme used for ERT (e.g., a human recombinant α-Gal A enzyme (rha-Gal A)), that is formulated together with an Active Site-Specific Chaperone (ASSC) for the α-Gal A enzyme (e.g., 1-deoxygalactonojirimycin (DGJ)). In some aspects, the ASSC is 1-deoxygalactonojirimycin (DGJ), or a pharmaceutically acceptable salt, ester or prodrug of 1-deoxygalactonojirimycin. In some aspects, the salt is hydrochloride salt (i.e. 1-deoxygalactonojirimycin-HCl). In some aspects, treating a subject with the co-formulation comprises administering the co-formulation to the subject such that the α-Gal A enzyme and ASSC are administered concurrently at the same time as part of the co-formulation.

The term “combination therapy” refers to any therapy wherein the results are enhanced as compared to the effect of each therapy when it is performed individually. The individual therapies in a combination therapy can be administered concurrently or consecutively.

Enhancement can include any improvement of the effect of the various therapies that can result in an advantageous result as compared to the results achieved by the therapies when performed alone. Enhanced effect and determination of enhanced effect can be measured by various parameters such as, but not limited to: temporal parameters (e.g., length of treatment, recovery time, long-term effect of the treatment or reversibility of treatment); biological parameters (e.g., cell number, cell volume, cell composition, tissue volume, tissue size, tissue composition); spatial parameters (e.g., tissue strength, tissue size or tissue accessibility) and physiological parameters (e.g., body contouring, pain, discomfort, recovery time or visible marks). Enhanced effect can include a synergistic enhancement, wherein the enhanced effect is more than the additive effects of each therapy when performed by itself. Enhanced effect can also include an additive enhancement, wherein the enhanced effect is substantially equal to the additive effect of each therapy when performed by itself. Enhanced effect can also include less than a synergistic effect, wherein the enhanced effect is lower than the additive effect of each therapy when performed by itself, but still better than the effect of each therapy when performed by itself.

The term “stabilize a proper conformation” refers to the ability of a compound or peptide or other molecule to associate with a wild-type protein, or to a mutant protein that can perform its wild-type function in vitro and in vivo, in such a way that the structure of the wild-type or mutant protein can be maintained as its native or proper form. This effect can manifest itself practically through one or more of (i) increased shelf-life of the protein; (ii) higher activity per unit/amount of protein; or (iii) greater in vivo efficacy. It can be observed experimentally through increased yield from the ER during expression; greater resistance to unfolding due to temperature increases (e.g., as determined in thermal stability assays), or the present of chaotropic agents, and by similar means.

As used herein, the term “active site” refers to the region of a protein that has some specific biological activity. For example, it can be a site that binds a substrate or other binding partner and contributes the amino acid residues that directly participate in the making and breaking of chemical bonds. Active sites in this application can encompass catalytic sites of enzymes, antigen biding sites of antibodies, ligand binding domains of receptors, binding domains of regulators, or receptor binding domains of secreted proteins. The active sites can also encompass transactivation, protein-protein interaction, or DNA binding domains of transcription factors and regulators.

As used herein, the term “active site-specific chaperone” refers to any molecule including a protein, peptide, nucleic acid, carbohydrate, etc. that specifically interacts reversibly with an active site of a protein and enhances formation of a stable molecular conformation. As used herein, “active site-specific chaperone” does not include endogenous general chaperones present in the ER of cells such as Bip, calnexin or calreticulin, or general, non-specific chemical chaperones such as deuterated water, DMSO, or TMAO.

The term “non-enzyme replacement therapy” refers to a therapy (e.g., Fabry disease therapy) that is not an enzyme replacement therapy. The non-enzyme replacement therapy can include small molecule therapy. Some emerging drug development strategies for small molecule therapy of Fabry disease include but are not limited to substrate reduction therapy (SRT), residual enzyme activation, GLA promoter activation, protein homeostasis regulation (proteostasis), and chemical chaperone therapy (CCT).

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms, “subject” and “patient” are used interchangeably herein.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., recombinant α-Gal A protein). For example, a 60 kg person and a 100 kg person would receive the same dose of an antibody (e.g., 12 mg of recombinant α-Gal A protein).

The term “weight-based dose” as referred to herein means that a dose that is administered to a patient is calculated based on the weight of the patient. For example, when a patient with 60 kg body weight requires 0.2 mg/kg of recombinant α-Gal A protein, one can calculate and use the appropriate amount of recombinant α-Gal A protein (i.e., 12 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

A “sample” or “biological sample” of the disclosure is of biological origin, in some aspects, such as from eukaryotic organisms. In some aspects, the sample is a human sample, but animal samples can also be used. Non-limiting sources of a sample for use in the present disclosure include solid tissue, biopsy aspirates, ascites, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example.

“Administering” refers to the physical introduction of a composition comprising a therapeutic agent to a subject, using any of the various methods and delivery systems known to those skilled in the art. Preferred routes of administration for recombinant α-Gal A protein or a gene expressing α-Gal A, include intravenous or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

The terms “once about every week,” “once about every two weeks,” or any other similar dosing interval terms as used herein mean approximate numbers. “Once about every week” can include every seven days ±one day, i.e., every six days to every eight days. “Once about every two weeks” can include every fourteen days ±three days, i.e., every eleven days to every seventeen days. Similar approximations apply, for example, to once about every three weeks, once about every four weeks, once about every five weeks, once about every six weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Disclosure

Provided herein are methods of treating Fabry disease in a human subject in need thereof comprising administering a therapy for Fabry disease to the subject, wherein the subject is identified as an anti-α-Galactosidase A (GLA) neutralizing antibody negative subject when a biological sample of the subject is analyzed, wherein the anti-GLA neutralizing antibody negative subject has a biological sample having lower than about 30% inhibition of α-Galactosidase A activity as measured by an anti-GLA neutralizing antibody assay.

In some aspects, the disclosure is directed to a method of identifying a human subject suitable for a therapy for Fabry disease comprising measuring the presence of an anti-GLA neutralizing antibody in a biological sample of the subject, wherein the subject suitable for a therapy is an anti-GLA neutralizing antibody negative subject when a biological sample of the subject is analyzed, wherein the anti-GLA neutralizing antibody negative subject has a biological sample having lower than about 30% inhibition of α-Galactosidase A activity as measured by an anti-GLA neutralizing antibody assay.

In some aspects, the disclosure is directed to a method of standardizing an anti-GLA neutralizing antibody assay comprising determining a GLA drug diluent concentration by measuring the effect of the GLA drug level on the inhibitory effect of an antibody designated as RP-01.

In some aspects, the disclosure is directed to a method of treating Fabry disease in a human subject in need thereof comprising administering a non-enzyme replacement therapy for Fabry disease to the subject, wherein the subject is identified as an anti-α-Galactosidase A (GLA) neutralizing antibody positive subject when a biological sample of the subject is analyzed, wherein the anti-GLA neutralizing antibody positive subject has a biological sample having higher than about 10% inhibition of α-Galactosidase A activity as measured by an anti-GLA neutralizing antibody assay.

In some aspects, the disclosure is directed to a method of identifying a human subject suitable for a non-enzyme replacement therapy for Fabry disease comprising measuring the presence of an anti-GLA neutralizing antibody in a biological sample of the subject, wherein the subject suitable for a therapy is an anti-GLA neutralizing antibody positive subject when a biological sample of the subject is analyzed, wherein the anti-GLA neutralizing antibody positive subject has a biological sample having higher than about 10% inhibition of α-Galactosidase A activity as measured by an anti-GLA neutralizing antibody assay.

In some aspects, the disclosure is directed to a method of identifying a human subject who is not eligible for an enzyme replacement therapy for Fabry disease comprising measuring the presence of an anti-GLA neutralizing antibody in a biological sample of the subject, wherein the sample having higher than about 10% inhibition of α-Galactosidase A activity as measured by the anti-GLA neutralizing antibody assay is identified as the anti-GLA neutralizing antibody positive sample, and wherein the subject not eligible for the enzyme replacement therapy for Fabry disease has the anti-GLA neutralizing antibody positive sample.

### IIA. Anti-GLA Neutralizing Antibodies

In some aspects, the disclosure is directed to methods of treating Fabry disease in a human subject with an enzyme or a non-enzyme replacement therapy for Fabry disease or identifying a human subject suitable for an enzyme or a non-enzyme replacement therapy for Fabry disease determined based on measuring the presence or absence of an anti-α-Galactosidase A (GLA) neutralizing antibody in a biological sample of the subject as measured by an anti-GLA neutralizing antibody assay.

In some aspects, the anti-GLA neutralizing antibody binds and inactivates (neutralizes) α-Gal A enzyme. In some aspects, if the anti-GLA neutralizing antibodies are present, the enzyme replacement therapy is directly inactivated (neutralized) by the anti-GLA neutralizing antibodies in the plasma.

In some aspects, if no anti-GLA neutralizing antibodies are present, the enzyme replacement therapy (e.g., recombinant α-Gal A enzyme) enters cells (e.g., endothelial cells) via the M6P receptor, leading to Gb3 clearance from lysosomes. In some aspects, if the anti-GLA neutralizing antibodies are present, they can neutralize the ERT activity by binding the enzyme (e.g., recombinant α-Gal A).

In some aspects, the anti-GLA neutralizing IgG antibody-tagged ERT molecules will be internalized and digested by macrophages. If more anti-GLA neutralizing antibodies than ERT are present, this can result in a decreased cellular Gb3 clearance. If the ERT dose exceeds the anti-GLA neutralizing antibody titers, more ERT can enter the lysosomes of target cells, resulting in increased Gb3 clearance. (Lenders et al., J Am Soc Nephrol 29:2265-2278 (2018).

The anti-GLA antibody neutralizing activity is described, for example, in Rombach et al., PLoS One 7: e47805 (2012); Lenders et al., J Am Soc Nephrol 27: 256-264 (2016); Smid et al., Mol Genet Metab 108:132-137 (2013).

In some aspects, the anti-GLA neutralizing antibody is an IgG antibody. In some aspects, the anti-GLA neutralizing antibody is an IgG4 antibody. In some aspects, the anti-GLA neutralizing antibody is an IgG2 antibody. In some aspects, the anti-GLA neutralizing antibody is an IgG1 antibody.

In some aspects, the anti-GLA neutralizing antibodies can develop within about one month, about two months, about three months, about four months, about five months, about six months, about seven months, about eight months, about nine months, about ten months, about eleven months, or within about twelve months of starting the enzyme replacement therapy.

In some aspects, the human subject that can develop the anti-GLA neutralizing antibodies, for example, a male patient with classical Fabry disease, as described in e.g., Van der Veen et al., Mol GenetMetab. 126(2):162-168 (2019); Wilcox et al., Mol Genet Metab. 105(3):443-449 (2012).

### IIB. Anti-GLA Neutralizing Antibody Assay

In some aspects, the disclosure is directed to methods of treating Fabry disease in a human subject with an enzyme or a non-enzyme replacement therapy for Fabry disease or identifying a human subject suitable for an enzyme or a non-enzyme replacement therapy for Fabry disease determined based on measuring the presence or absence of an anti-α-Galactosidase A (GLA) neutralizing antibody in a biological sample of the subject as measured by an anti-GLA neutralizing antibody assay.

In some aspects, the anti-GLA neutralizing antibody assay, as described in Example 1 below, determines the presence of anti-GLA neutralizing antibodies by assessing the neutralizing capacity of human serum on GLA activity. In some aspects, the results are determined by measuring 4-methylumbelliferone (4-MU) product resulting from cleavage of an artificial substrate, 4-methylumbelliferyl α-Dgalactopyranoside (4-MU-α-Gal). Any anti-GLA neutralizing antibody present in the human serum will bind to the GLA and prevent the cleavage of 4-MU from 4-methylumbelliferyl α-D-galactopyranoside (4-MU-α-Gal) substrate. This reduction in Relative Fluorescence Unit (RFU) signal is directly proportional to the amount of the anti-GLA neutralizing antibody present in the human serum. 101311 In some aspects, biological sample is a serum sample, which is diluted at MRD15 or lower. In some aspects, the serum sample is diluted at MRD10 (1:5 in assay buffer (e.g., M Citric Acid, 0.2 M Sodium Phosphate and 0.05% Triton X-100, pH 4.6±0.1) followed by 1:2 in 2X drug diluent (e.g., 40 ng/mL reconstituted GLA protein in assay buffer)).

In some aspects, the GLA is at a concentration of less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about 20 ng/ml, or less than about 10 ng/ml. In some aspects, the GLA is at a concentration of about 20 ng/ml.

In some aspects, the serum sample and the GLA mixture are incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

In some aspects, the serum sample and the GLA mixture are incubated for a duration between about 1 and about 15 hours, between about 2 and about 14 hours, between about 3 and about 13 hours, between about 4 and about 12 hours, between about 5 and about 11 hours, between about 6 and about 10 hours, or between about 7 and about 9 hours.

In some aspects, the serum sample is mixed with a reaction mix. In some aspects, the reaction mix comprises a substrate and an inhibitor. In some aspects, the substrate comprises 4-methylumbelliferyl (4-MU)-α-D-galactopyranoside. In some aspects, the inhibitor comprises N-Acetylgalactosamine (GALNAc).

In some aspects, the substrate is at a concentration of at least about 1.1 mM, at least about 1.2 mM, at least about 1.3 mM, at least about 1.4 mM, at least about 1.5 mM, at least about 1.6 mM, at least about 1.7 mM, at least about 1.8 mM, at least about 1.9 mM, at least about 2 mM, at least about 2.1 mM, at least about 2.2 mM, at least about 2.3 mM, at least about 2.4 mM, at least about 2.5 mM, at least about 2.6 mM, at least about 2.7 mM, at least about 2.8 mM, at least about 2.9 mM or at least about 3 mM. In some aspects, the substrate is at a concentration of at least about 2.5 mM.

In some aspects, the inhibitor is at a concentration of less than about 200 mM, less than about 195 mM, less than about 190 mM, less than about 185 mM, less than about 180 mM, less than about 175 mM, less than about 170 mM, less than about 165 mM, less than about 160 mM, less than about 155 mM, less than about 150 mM, less than about 145 mM, less than about 140 mM, less than about 135 mM, less than about 130 mM, less than about 125 mM, less than about 120 mM, less than about 115 mM, or less than about 110 mM. In some aspects, the inhibitor is at a concentration of at least about 125 mM.

In some aspects, the reaction mix and the serum sample are mixed in a high throughput plate. In some aspects, the reaction mix and the serum sample mixture are incubated at room temperature at revolutions per minute (RPM) 400.

In some aspects, the method disclosed herein further comprising adding a stop buffer to the mixture after incubation. In some aspects, the incubation period is at least about 30 minutes, at least about 35 minutes, at least about 40 minutes, at least about 45 minutes, at least about 50 minutes, at least about 55 minutes, at least about 60 minutes, at least about 65 minutes, at least about 70 minutes, at least about 75 minutes, or at least about 80 minutes. In some aspects, the incubation period is at least about 60 minutes.

In some aspects, the stop buffer comprises glycine. In some aspects, the stop buffer is at a volume of less than about 1 mL, less than about 900 uL, less than about 800 uL, less than about 700 uL, less than about 600 uL, less than about 500 uL, less than about 400 uL, less than about 300 uL, less than about 200 uL, or less than about 100 uL. In some aspects, the stop buffer is at a volume of about 100 uL.

In some aspects, human biological samples having percent (%) inhibition equal to or greater than the cut-off point are identified as the anti-GLA neutralizing antibody positive, while those below the cut-off point are considered the anti-GLA neutralizing antibody negative.

In some aspects, the anti-GLA neutralizing antibody negative subject as described herein has a biological sample having lower than about 30% inhibition of α-Galactosidase A activity as measured by an anti-GLA neutralizing antibody assay. In some aspects, the anti-GLA neutralizing antibody negative sample as described herein has about 29%, about 28%, about 27%, about 26%, about 25%, about 24%, about 23%, about 22%, about 21%, or about 20% inhibition of α-Galactosidase A activity. In some aspects, the anti-GLA neutralizing antibody negative sample as described herein has lower than about 15%, about 16%, about 17% about 18%, about 19%, about 20%, about 21%, about 22%, about 23%, about 24%, about 25%, about 26%, about 27%, about 28%, about 29%, or about 30% inhibition of α-Galactosidase A activity.

In some aspects, the anti-GLA neutralizing antibody negative sample has lower than about 27% inhibition of α-Galactosidase A activity. In some aspects, the anti-GLA neutralizing antibody negative sample has lower than about 20% inhibition of α-Galactosidase A activity. In some aspects, the anti-GLA neutralizing antibody negative sample has lower than about 15% inhibition of α-Galactosidase A activity. In some aspects, the anti-GLA neutralizing antibody negative sample has lower than about 10% inhibition of α-Galactosidase A activity. In some aspects, the anti-GLA neutralizing antibody negative sample has lower than about 1% inhibition of α-Galactosidase A activity. In some aspects, the anti-GLA neutralizing antibody negative sample has no inhibition of α-Galactosidase A activity. In some aspects, the anti-GLA neutralizing antibody negative sample has about 1% to about 10% inhibition of α-Galactosidase A activity. In some aspects, the anti-GLA neutralizing antibody negative sample has about 10% to about 20% inhibition of α-Galactosidase A activity. In some aspects, the anti-GLA neutralizing antibody negative sample has about 20% to about 30% inhibition of α-Galactosidase A activity.

In some aspects, the anti-GLA neutralizing antibody negative sample has 27% inhibition of α-Galactosidase A activity. In some aspects, the anti-GLA neutralizing antibody negative sample has 26% inhibition of α-Galactosidase A activity. In some aspects, the anti-GLA neutralizing antibody negative sample has 25% inhibition of α-Galactosidase A activity. In some aspects, the anti-GLA neutralizing antibody negative sample has 24% inhibition of α-Galactosidase A activity. In some aspects, the anti-GLA neutralizing antibody negative sample has 23% inhibition of α-Galactosidase A activity. In some aspects, the anti-GLA neutralizing antibody negative sample has 22% inhibition of α-Galactosidase A activity. In some aspects, the anti-GLA neutralizing antibody negative sample has 21% inhibition of α-Galactosidase A activity. In some aspects, the anti-GLA neutralizing antibody negative sample has 20% inhibition of α-Galactosidase A activity. In some aspects, the anti-GLA neutralizing antibody negative sample has 19% inhibition of α-Galactosidase A activity. In some aspects, the anti-GLA neutralizing antibody negative sample has 18% inhibition of α-Galactosidase A activity. In some aspects, the anti-GLA neutralizing antibody negative sample has 17% inhibition of α-Galactosidase A activity. In some aspects, the anti-GLA neutralizing antibody negative sample has 16% inhibition of α-Galactosidase A activity. In some aspects, the anti-GLA neutralizing antibody negative sample has 15% inhibition of α-Galactosidase A activity. In some aspects, the anti-GLA neutralizing antibody negative sample has 20% inhibition of α-Galactosidase A activity. In some aspects, the anti-GLA neutralizing antibody negative sample has 14% inhibition of α-Galactosidase A activity. In some aspects, the anti-GLA neutralizing antibody negative sample has 13% inhibition of α-Galactosidase A activity. In some aspects, the anti-GLA neutralizing antibody negative sample has 12% inhibition of α-Galactosidase A activity. In some aspects, the anti-GLA neutralizing antibody negative sample has 11% inhibition of α-Galactosidase A activity. In some aspects, the anti-GLA neutralizing antibody negative sample has 10% inhibition of α-Galactosidase A activity. In some aspects, the anti-GLA neutralizing antibody negative sample has 9% inhibition of α-Galactosidase A activity. In some aspects, the anti-GLA neutralizing antibody negative sample has 8% inhibition of α-Galactosidase A activity. In some aspects, the anti-GLA neutralizing antibody negative sample has 7% inhibition of α-Galactosidase A activity. In some aspects, the anti-GLA neutralizing antibody negative sample has 6% inhibition of α-Galactosidase A activity. In some aspects, the anti-GLA neutralizing antibody negative sample has 5% inhibition of α-Galactosidase A activity. In some aspects, the anti-GLA neutralizing antibody negative sample has 4% inhibition of α-Galactosidase A activity. In some aspects, the anti-GLA neutralizing antibody negative sample has 3% inhibition of α-Galactosidase A activity. In some aspects, the anti-GLA neutralizing antibody negative sample has 2% inhibition of α-Galactosidase A activity. In some aspects, the anti-GLA neutralizing antibody negative sample has 1% inhibition of α-Galactosidase A activity.

In some aspects, the anti-GLA neutralizing antibody positive subject as described herein has a biological sample having higher than about 10% inhibition of α-Galactosidase A activity as measured by an anti-GLA neutralizing antibody assay. In some aspects, the anti-GLA neutralizing antibody positive sample as described herein has about 27%, about 28%, about 29%, about 30%, about 31%, about 32%, about 33%, about 34%, about 35%, about 36%, about 37%, about 38%, about 39%, or about 40% signal inhibition.

In some aspects, the anti-GLA neutralizing antibody positive sample has 28% inhibition of α-Galactosidase A activity. In some aspects, the anti-GLA neutralizing antibody positive sample has 29% inhibition of α-Galactosidase A activity. In some aspects, the anti-GLA neutralizing antibody positive sample has 30% inhibition of α-Galactosidase A activity. In some aspects, the anti-GLA neutralizing antibody positive sample has 31% inhibition of α-Galactosidase A activity. In some aspects, the anti-GLA neutralizing antibody positive sample has 32% inhibition of α-Galactosidase A activity. In some aspects, the anti-GLA neutralizing antibody positive sample has 33% inhibition of α-Galactosidase A activity. In some aspects, the anti-GLA neutralizing antibody positive sample has 34% inhibition of α-Galactosidase A activity. In some aspects, the anti-GLA neutralizing antibody positive sample has 35% inhibition of α-Galactosidase A activity. In some aspects, the anti-GLA neutralizing antibody positive sample has 36% inhibition of α-Galactosidase A activity. In some aspects, the anti-GLA neutralizing antibody positive sample has 37% inhibition of α-Galactosidase A activity. In some aspects, the anti-GLA neutralizing antibody positive sample has 38% inhibition of α-Galactosidase A activity. In some aspects, the anti-GLA neutralizing antibody positive sample has 39% inhibition of α-Galactosidase A activity. In some aspects, the anti-GLA neutralizing antibody positive sample has 40% inhibition of α-Galactosidase A activity.

### IIC. Standardization of an Anti-GLA Neutralizing Antibody Assay

In some aspects, the disclosure is directed to a method of standardizing an anti-GLA neutralizing antibody assay comprising determining a GLA drug diluent concentration by measuring the effect of the GLA drug level on the inhibitory effect of the positive control antibody (e.g., an antibody designated as RP-01).

In some aspects, the positive control antibody includes, but is not limited to the RP-01 antibody. In some aspects, the RP-01 is a polyclonal antibody. The RP-01 positive control antibody was generated as described in Example 2 below.

In some aspects, the standardization comprises determining a GLA drug diluent concentration by measuring the effect of the GLA drug level on the inhibitory effect of the RP-01 antibody. In some aspects, the GLA drug diluent concentration is less than about 100 ng/ml, less than about 90 ng/ml, less than about 80 ng/ml, less than about 70 ng/ml, less than about 60 ng/ml, less than about 50 ng/ml, less than about 40 ng/ml, less than about ng/ml, less than about 20 ng/ml, or less than about 10 ng/ml. In some aspects, the GLA drug diluent concentration is about 40 ng/ml. In some aspects, the GLA drug diluent concentration is about 20 ng/ml.

In some aspects, the inhibitory effect of the RP-01 antibody is represented as % inhibition as measured by the anti-GLA neutralizing antibody assay using different concentrations of the RP-01 antibody. In some aspects, the inhibitory effect of RP-01 is represented as about 20%, about 21%, about 22%, about 23%, about 24%, about 25%, about 26%, about 27%, about 28%, about 29%, or about 30% inhibition of α-Galactosidase A activity at about 50 ug/ml RP-01 concentration. In some aspects, the inhibitory effect of RP-01 is represented as about 25% inhibition of α-Galactosidase A activity at about 50 ug/ml RP-01 concentration.

In some aspects, the inhibitory effect of RP-01 is represented as about 30%, about 31%, about 32%, about 33%, about 34%, about 35%, about 36%, about 37%, about 38%, about 39%, or about 40% inhibition of α-Galactosidase A activity at about 100 ug/ml RP-01 concentration. In some aspects, the inhibitory effect of RP-01 is represented as about 37% inhibition of α-Galactosidase A activity at about 100 ug/ml RP-01 concentration.

In some aspects, the inhibitory effect of RP-01 is represented as about 40%, about 41% RFU, about 42%, about 43%, about 44%, about 45%, about 46%, about 47%, about 48%, about 49%, or about 50% inhibition of α-Galactosidase A activity at about 150 ug/ml RP-01 concentration. In some aspects, the inhibitory effect of RP-01 is represented as about 48% inhibition of α-Galactosidase A activity at about 150 ug/ml RP-01 concentration.

In some aspects, the GLA drug has a minimal required dilution (MRD) about 1 fold, about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, about 41 fold, about 42 fold, about 43 fold, about 44 fold, about 45 fold, about 46 fold, about 47 fold, about 48 fold, about 49 fold, about 50 fold, about 51 fold, about 52 fold, about 53 fold, about 54 fold, about 55 fold, about 56 fold, about 57 fold, about 58 fold, about 59 fold, about 60 fold, about 61 fold, about 62 fold, about 63 fold, about 64 fold, about 65 fold, about 66 fold, about 67 fold, about 68 fold, about 69 fold, about 70 fold, about 71 fold, about 72 fold, about 73 fold, about 74 fold, about 75 fold, about 76 fold, about 77 fold, about 78 fold, about 79 fold, about 80 fold, about 81 fold, about 82 fold, about 83 fold, about 84 fold, about 85 fold, about 86 fold, about 87 fold, about 88 fold, about 89 fold, about 90 fold, about 91 fold, about 92 fold, about 93 fold, about 94 fold, about 95 fold, about 96 fold, about 97 fold, about 98 fold, about 99 fold, or about 100 fold.

In some aspects, the RP-01 antibody has binding affinity (K_{D}) of less than 2.6×10⁻¹⁰ M, less than 2.5×10⁻¹⁰ M, less than 2.0×10⁻¹⁰ M, less than 1.5×10⁻¹⁰ M, less than 1.0×10⁻¹⁰ M, less than 9×10⁻¹¹ M, less than 8×10⁻¹¹ M, less than 7×10⁻¹¹ M, less than 6×10⁻¹¹ M, less than 5×10⁻¹¹ M, less than 4×10⁻¹¹ M, less than 3×10⁻¹¹ M, less than 2×10⁻¹¹ M, less than 1×10⁻¹¹ M, less than 9×10⁻¹² M, less than 8×10⁻¹² M, less than 7×10⁻¹² M, less than 6×10⁻¹² M, less than 5×10⁻¹² M, less than 4×10⁻¹² M, less than 3×10⁻¹² M, less than 2×10⁻¹² M, less than 1×10⁻¹² M, less than 9×10⁻¹³ M, or less than 8×10⁻¹³ M, when determined by, e.g., immunoassays (e.g., ELISA) surface plasmon resonance (SPR) technology in a BIACORE™ 2000 instrument using the predetermined antigen, or BLI (Bio-layer interferometry).

### III. Fabry Disease Therapies

In some aspects, the disclosure is directed to methods of treating Fabry disease in a human subject with an enzyme or a non-enzyme replacement therapy for Fabry disease or identifying a human subject suitable for an enzyme or a non-enzyme replacement therapy for Fabry disease determined based on measuring the presence or absence of an anti-α-Galactosidase A (GLA) neutralizing antibody in a biological sample of the subject as measured by an anti-GLA neutralizing antibody assay described herein.

### III.A Enzyme Replacement Therapy

In some aspects, the therapy for Fabry disease is an enzyme replacement therapy.

In some aspects, the subject has been administered an enzyme replacement therapy for Fabry disease prior to administering the enzyme replacement therapy and/or measuring the presence or absence of an anti-α-Galactosidase A (GLA) neutralizing antibody in a biological sample of the subject as described herein (“pre-treatment”).

In some aspects, the enzyme replacement therapy and/or the pre-treatment comprises a recombinant a Galactosidase A (GLA) protein or a gene expressing GAL. In some aspects, the enzyme replacement therapy and/or the pre-treatment comprises agalsidase alpha and/or beta or a gene expressing agalsidase alpha and/or beta. In some aspects, the enzyme replacement therapy comprises administering agalsidase alfa (Replagalg, Shire Human Genetic Therapies), agalsidase beta (Fabrazyme®; Sanofi Genzyme), pegunigalsidase alfa (PRX-102; Protalix BioTherapeutics), or any combination thereof. These forms of ERT are intended to compensate for a patient's inadequate α-Gal A activity with a recombinant form of the enzyme, administered intravenously. ERT has been demonstrated to reduce Gb3 deposition in capillary endothelium of the kidney and some other cell types. While ERT is effective in many settings, the treatment also has limitations. ERT has not been demonstrated to decrease the risk of stroke, cardiac muscle responds slowly, and Gb3 elimination from some of the cell types of the kidneys is limited. Some patients develop immune reactions to ERT. See e.g., U.S. Pat. No. 10,155,027.

In some aspects, about 0.2 mg/kg body weight of agalsidase alfa is infused every 2 weeks as an intravenous infusion.

In some aspects, about 0.3 mg/kg body weight of agalsidase beta is infused every 2 weeks as an intravenous infusion. In some aspects, about 1 mg/kg body weight of agalsidase beta is infused every 2 weeks as an intravenous infusion.

In some aspects, saturating the higher anti-GLA neutralizing antibody levels in a subject via higher ERT dosing can provide clinical benefit (e.g., decrease plasma lyso-Gb3 levels). See e.g., Lenders et al., Orphanet Journal of Rare Diseases, 13(171) (2018).

In some aspects, the enzyme replacement therapy and/or the pre-treatment comprises a gene therapy. In some aspects, the gene therapy comprises a vector encoding the enzyme. In some aspects, the vector is a viral vector. In some aspects, the viral vector comprises an adeno-associated virus (AAV) vector or a lentiviral vector. In some aspects, the gene therapy comprises administering ST-920 (Sangamo Therapeutics, Inc.), AVR-RD-01 (AvroBio), FLT-190 (Freeline Therapeutics), or any combination thereof. ST-920 comprises an AAV vector carrying a GLA gene construct driven by a liver-specific promoter. ST-920 gene therapy is designed to enable a patient's liver to produce a long-lasting and continuous supply of the α-Gal A enzyme. (ClinicalTrials.gov Identifier: NCT04046224). AVR-RD-01 drug product comprises autologous CD34+ cell-enriched fraction that contains cells transduced with Lentiviral Vector/alpha-galactosidase A (AGA) encoding for the human AGA complementary deoxyribonucleic acid (cDNA) sequence. (ClinicalTrials.gov; Identifier: NCT03454893). FLT190 is a single stranded (ss) AAV gene therapy construct with a codon-optimized human GLA cDNA driven by a liver specific promotor (FRE1), pseudotyped with AAV8 capsid (ssAAV8-FRE1-GLAco). (Nephron Clinical Practice, Abstracts: 6th Update on Fabry Disease: Biomarkers, Progression and Treatment Opportunities, May 26-28, 2019, Prague, Czech Republic).

In some aspects, the gene therapy comprises a vector encoding the enzyme. In some aspects, the vector comprises an mRNA encoding a human GLA protein or agalsidase alpha and/or beta, as described in e.g., U.S. Pat. No. 9,308,281. In some aspects, the mRNA can comprise one or more modifications that confer stability to the mRNA (e.g., compared to a wild-type or native version of the mRNA) and can also comprise one or more modifications relative to the wild-type which correct a defect implicated in the associated aberrant expression of the protein. For example, the nucleic acids of the disclosure can comprise modifications to one or both of the 5′ and 3′ untranslated regions. Such modifications can include, but are not limited to, the inclusion of a partial sequence of a cytomegalovirus (CMV) immediate-early 1 (IE1) gene, a poly A tail, a Cap1 structure or a sequence encoding human growth hormone (hGH)). In some aspects, the mRNA is modified to decrease mRNA immunogenecity.

In some aspects, the gene therapy is delivered by a transfer vehicle. In some aspects, the transfer vehicle is a liposomal transfer vehicle, e.g., a lipid nanoparticle, as described in U.S. Pat. No. 9,308,281. In some aspects, the mRNA encoding a human GLA protein or agalsidase alpha and/or beta is formulated in a liposomal transfer vehicle to facilitate delivery to the target cell. Contemplated transfer vehicles can comprise one or more cationic lipids, non-cationic lipids, and/or PEG-modified lipids. For example, the transfer vehicle can comprise at least one of the following cationic lipids: C12-200, DLin-KC2-DMA, DODAP, HGT4003, ICE, HGT5000, and HGT5001. In some aspects, the transfer vehicle comprises cholesterol (chol) and/or a PEG-modified lipid. In some aspects, the transfer vehicles comprises DMG-PEG2K. In some aspects, the transfer vehicle comprises one of the following lipid formulations: C12-200, DOPE, chol, DMG-PEG2K; DODAP, DOPE, cholesterol, DMG-PEG2K; HGT5000, DOPE, chol, DMG-PEG2K, HGT5001, DOPE, chol, and DMG-PEG2K.

In some aspects, the enzyme replacement therapy and/or the pre-treatment comprises administering Galafold® (migalastat; Amicus Therapeutics). Galafold® is an alpha-galactosidase A (alpha-Gal A) pharmacological chaperone. In some aspects, 123 mg of Galafold® is administered orally once every other day at the same time of day, as described in e.g., Lenders et al., J Am Soc Nephrol, 29:2265-2278 (2018).

### III.B Enzyme Replacement Therapy and an Active Site-Specific Chaperone

In some aspects, the disclosure is directed to methods of treating Fabry disease in a human subject with α-Gal A (e.g., rha-Gal A) in combination with an active site-specific chaperone (ASSC) for the α-Gal A, e.g., migalastat (1-deoxygalactonojirimycin (DGJ)), as described in e.g., U.S. Pat. No. 10,155,027.

In some aspects, the disclosure provides for combination therapy of α-Gal A (e.g. rha-Gal A ERT) and an ASSC for the α-Gal A enzyme (e.g., (DGJ)). In some aspects, the α-Gal A and ASSC are co-formulated together and administered to a subject concurrently as a co-formulation. In some aspects, the ASSC 1-deoxygalactonojirimycin is co-formulated with α-Gal A as a pharmaceutical composition. Such a composition can enhance stability of α-Gal A both during storage (i.e., in vitro) and in vivo after administration to a subject, thereby increasing circulating half-life, tissue uptake, and resulting in increased therapeutic efficacy of α-Gal A (e.g., increasing the reduction of tissue GL-3 levels). In some aspects, the route of administration is intravenous. Administration can be by periodic injections of a bolus of the preparation, or as a sustained release dosage form over long periods of time, such as by intravenous administration, for example, from a reservoir which is external (e.g., an IV bag).

The co-formulation suitable for intravenous administration use include sterile aqueous solutions or dispersions and sterile powders for the extemporaneous preparation of sterile injectable solutions or dispersion. in some aspects, the form is sterile and fluid to the extent that easy syringability exists. In some aspects, it is stable under the conditions of manufacture and storage and preserved against the contaminating action of microorganisms such as bacteria and fungi. In some aspects, the co-formulation comprises a carrier such as a solvent or dispersion medium containing, for example, water, ethanol, polyol glycerol, propylene glycol, and polyethylene glycol, and the like), suitable mixtures thereof, and vegetable oils, The proper fluidity can be maintained, for example, by the use of a coating such as lecithin, by the maintenance of the required particle size in the case of dispersion, and by the use of surfactants. The preventions of the action of microorganisms can be brought about by various antibacterial and antifungal agents (e.g., parabens, chlorobutanol, phenol, benzyl alcohol, sorbic acid, and the like).

In some aspects, isotonic agents, for example, sugars or sodium chloride are added. Prolonged absorption of the injectable compositions can be brought about by the use in the compositions of agents delaying absorption, for example, aluminum monosterate and gelatin. Sterile injectable solutions can be prepared by incorporating the α-Gal A and ASSC (e.g., DGJ) in the required amounts in the appropriate solvent with various other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

In some aspects, the co-formulation can contain an excipient. Pharmaceutically acceptable excipients which can be included in the co-formulation are buffers such as citrate buffer, phosphate buffer, acetate buffer, and bicarbonate buffer, amino acids, urea, alcohols, ascorbic acid, phospholipids; proteins, such as serum albumin, collagen, and gelatin, salts (such as EDTA, EGTA, and sodium chloride), liposomes, polyvinylpyrollidone, sugars (such as dextran, mannitol, sorbitol, and glycerol), propylene glycol, and polyethylene glycol (e.g., PEG-4000, PEG-6000), glycerol, glycine (or other amino acids), and lipids. Buffer systems for use with the co-formulations can include citrate, acetate, bicarbonate, and phosphate buffers.

The co-formulation can also contain a non-ionic detergent. Non-ionic detergents include, but are not limited to, Polysorbate 20, Polysorbate 80, Triton X-100, Triton X-114, Nonidet P-40, Octyl α-glucoside, Octyl (3-glucoside, Brij 35, Pluronic, and Tween 20.

For lyophilization of protein and chaperone preparations, the protein concentration can be about 0.1 mg/mL to about 10 mg/mL. Bulking agents, such as glycine, mannitol, albumin, and dextran, can be added to the lyophilization mixture. In addition, possible cryoprotectants, such as disaccharides, amino acids, and PEG, can be added to the lyophilization mixture. Any of the buffers, excipients, and detergents listed above, can also be added.

In some aspects, the co-formulation comprises α-Gal A at a concentration of between about 0.05 and about 100 μM, between about 0.1 and about 75 μM, between about 0.2 and about 50 μM, between about 0.3 and about 40 μM, between about 0.4 and about 30 μM, between about 0.5 and about 20 μM, between about 0.6 and about 15 μM, between about 0.7 and about 10 μM, between about 0.8 and about 9 μM, between about 0.9 and about 8 μM, between about 1 and about 7 μM, between about 2 and about 6 μM, or between about 3 and about 5 μM.

In some aspects, the co-formulation comprises α-Gal A at a concentration of about 0.2, 0.3, 0.4, 0.5, 0.6, 0.7, 0.8, 0.9, 1, 1.1, 1.2, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 μM.

In some aspects, the co-formulation comprises α-Gal A at a concentration of between about 0.0025 and about 5 mg/ml, between about 0.005 and about 4.5 mg/ml, between about 0.025 and about 4 mg/ml, between about 0.05 and about 3.5 mg/ml, between about 0.25 and about 3 mg/ml, between about 0.5 and about 2.5 mg/ml, between about 0.75 and about 2 mg/ml, or between about 1 and about 1.5 mg/ml.

In some aspects, the co-formulation comprises DGJ at a concentration of between about 10 and about 25,000 μM, between about 50 and about 20,000 μM, between about 100 and about 15,000 μM, between about 150 and about 10,000 μM, between about 200 and about 5,000 μM, between about 250 and about 1,500 μM, between about 300 and about 1,000 μM, between about 350 and about 550 μM, or between about 400 and about 500 μM.

In some aspects, the co-formulation comprises DGJ at a concentration of between about 0.002 and about 5 mg/ml, between about 0.005 and about 4.5 mg/ml, between about and about 4 mg/ml, between about 0.05 and about 3.5 mg/ml, between about 0.2 and about 3 mg/ml, between about 0.5 and about 2.5 mg/ml, or between about 1 and about 2 mg/ml.

In some aspects, the co-formulation comprises DGJ at a concentration of about 50, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000, or 20000 μM.

In some aspects, the α-Gal A enzyme and DGJ are combined to create a co-formulation for administration to a subject, wherein the dosage of α-Gal A enzyme of the co-formulation administered to the subject is between about 0.05 and about 10 mg/kg, between about 0.1 and about 5 mg/kg, between about 0.2 and about 4 mg/kg, between about and about 3 mg/kg, between about 0.4 and about 2 mg/kg, between about 0.5 and about 1.5 mg/kg, or between about 0.5 and about 1 mg/kg.

In some aspects, the dosage of α-Gal A enzyme of the co-formulation administered to the subject is about 0.05, 0.1, 0.2, 0.3, 0.4, 0.5, 1, 1.5, 2, 2.5, 3, 3.5, 4, 4.5, or 5 mg/kg.

In some aspects, the α-Gal A enzyme and DGJ are combined to create a co-formulation for administration to a subject, wherein the dosage of DGJ of the co-formulation administered to the subject is between about 0.05 and 20 mg/kg, between about 0.1 and about 15 mg/kg, between about 0.2 and about 10 mg/kg, between about 0.3 and about 10 mg/kg, between about 0.4 and about 9 mg/kg, between about 0.5 and about 8 mg/kg, between about 0.6 and about 7 mg/kg, between about 0.7 and about 6 mg/kg, between about 0.8 and about 5 mg/kg, between about 0.9 and about 4 mg/kg, between about 1 and about 3 mg/kg, or between about 1.5 and about 2 mg/kg.

In some aspects, the dosage of DGJ of the co-formulation administered to the subject is about 0.05, 0.1, 0.2, 0.3, 0.4, 0.5, 0.6, 0.7, 0.8, 0.9, 1, 2, 3, 4, 5, 6, 7, 8, 9 or 10 mg/kg.

In some aspects, the co-formulation of α-Gal A and DGJ can be administered intravenously to a subject in an amount effective to achieve a plasma AUC concentration of between about 0.5 and 10-fold, between about 1 and about 8-fold, between about 1.5 and about 6-fold, between about 2 and about 5.5-fold, between about 2.5 and about 5-fold, or between about 3 and about 4.5-fold of the plasma AUC concentration achieved when α-Gal A is administered to a subject in the same dosage as the co-formulation, but in the absence of DGJ.

As used herein, the term “AUC” represents a mathematical calculation to evaluate the body's total exposure over time to a given drug. In a graph plotting how concentration in the blood after dosing, the drug concentration variable lies on the y-axis and time lies on the x-axis. The area between a drug concentration curve and the x-axis for a designated time interval is the AUC. AUCs are used as a guide for dosing schedules and to compare different drugs' availability in the body.

In some aspects, the co-formulation of α-Gal A and DGJ can be administered intravenously to a subject in an amount effective to achieve a level of α-Gal A tissue uptake of between about 0.5 and 10-fold, between about 1 and about 8-fold, between about 1.5 and about 6-fold, between about 2 and about 5.5-fold, between about 2.5 and about 5-fold, or between about 3 and about 4.5-fold of the level of α-Gal A tissue uptake achieved when α-Gal A is administered to a subject in the same dosage as the co-formulation, but in the absence of DGJ.

Delivery of the co-formulation can be continuous over a pre-selected administration period ranging from several hours, one to several weeks, one to several months, or up to one or more years. In some aspects, the dosage form is one that is adapted for delivery of α-Gal A over an extended period of time. Such delivery devices can be adapted for administration of α-Gal A for several hours (e.g., 2 hours, 12 hours, or 24 hours to 48 hours or more), to several days (e.g., 2 to 5 days or more, from about 100 days or more), to several months or years. In some aspects, the device is adapted for delivery for a period ranging from about 1 month to about 12 months or more. The α-Gal A delivery device can be one that is adapted to administer α-Gal A to an individual for a period of, e.g., from about 2 hours to about 72 hours, from about 4 hours to about 36 hours, from about 12 hours to about 24 hours; from about 2 days to about 30 days, from about 5 days to about 20 days, from about 7 days to about 100 days or more, from about 10 days to about 50 days; from about 1 week to about 4 weeks; from about 1 month to about 24 months or more, from about 2 months to about 12 months, from about 3 months to about 9 months, or other ranges of time, including incremental ranges, within these ranges, as needed.

In some aspects, a dose of α-Gal A present in a co-formulation with DGJ is the intravenously administered once per day, once every two days, once every three days, once every four days, once every five days, or once every six days. In some aspects, the dose does not result in a toxic level of α-Gal A in the liver of the individual. In some aspects, the co-formulation composition of α-Gal A and DGJ is administered in a sufficient dose to result in a peak concentration of α-Gal A in tissues of the subject, within about 24 hours after the administration of the dose. In some aspects, the co-formulation composition is administered in a sufficient dose to result in a peak concentration of α-Gal A in tissues of the subject within between about 0.2 to about 50 hours, between about 0.2 to about 24 hours, between about 0.2 to about 5 hours, between about 0.2 to about 1 hour, between about 0.2 to about 0.5 hour, or about 40, 30, 20, 10, 5, 1, 0.5 or fewer hours after the administration of the dose. In some aspects, the co-formulation is administered as a single-dose. In some aspects, the co-formulation is administered as a multi-dose.

III.0 Treatment with Intravenous 1-deoxygalactonojirimycin (DGJ)

Also provided herein are methods of using intravenous administration of DGJ or salts thereof for the treatment of Fabry disease. In some aspects, a salt such as DGJ HCl is used.

Administration can be by periodic injections of a bolus of the preparation, or as a sustained release dosage form over long periods of time, such as by intravenous administration, for example, from a reservoir which is external (e.g., an IV bag).

In some aspects, the dosage of DGJ of salt thereof administered to the subject is between about 0.05 and 20 mg/kg, between about 0.1 and about 15 mg/kg, between about and about 10 mg/kg, between about 0.3 and about 10 mg/kg, between about 0.4 and about 9 mg/kg, between about 0.5 and about 8 mg/kg, between about 0.6 and about 7 mg/kg, between about 0.7 and about 6 mg/kg, between about 0.8 and about 5 mg/kg, between about and about 4 mg/kg, between about 1 and about 3 mg/kg, or between about 1.5 and about 2 mg/kg.

In some aspects, the dosage of DGJ or salt thereof administered to the subject is about 0.05, 0.1, 0.2, 0.3, 0.4, 0.5, 0.6, 0.7, 0.8, 0.9, 1, 2, 3, 4, 5, 6, 7, 8, 9 or 10 mg/kg.

In some aspects, the methods described herein include administering to an individual, for example, by intravenous administration, a dose of DGJ or salt thereof, wherein the dose is administered once per day, once every two days, once every three days, once every four days, once every five days, once every six days, once a week or once every two weeks. In some aspects, the DGJ is administered as a single-dose. In some aspects, the DGJ is administered as a multi-dose.

### III.D Non-Enzyme Replacement Therapy

In some aspects, the therapy for Fabry disease is an enzyme replacement therapy.

In some aspects, the non-enzyme replacement therapy comprises small molecule therapy. Some emerging drug development strategies for small molecule therapy of Fabry disease include but are not limited to substrate reduction therapy (SRT), residual enzyme activation, GLA promoter activation, protein homeostasis regulation (proteostasis), and chemical chaperone therapy (CCT), as described in e.g., Motabar et al., Curr Chem Genomics 4: 50-56 (2010).

In some aspects, small molecule therapy comprises administering lucerastat (Idorsia Pharmaceuticals Ltd), venglustat (Sanofi Genzyme), or apabetalone (development codes RVX 208, RVX-208, and RVX000222; Resverlogix Corp.), or any combination thereof.

### III.E Other Therapeutic Options in Patients with Anti-GLA Neutralizing Antibodies

The conflicting outcomes in studies reporting the effect of anti-GLA neutralizing antibodies on clinical symptoms and manifestations in patients receiving the enzyme replacement therapy demonstrate the need to determine individual antibody titers, especially because the anti-GLA neutralizing antibody titers can be supersaturated by appropriate enzyme doses at some point of infusion. The easiest method to overcome the anti-GLA neutralizing antibody titers and to increase the enzyme replacement therapy efficacy thus might be to increase infused dosages. However, the maximum approved doses of agalsidase-α and agalsidase-0 per kg body weight are limited, and using higher dosages of these expensive drugs would be very costly. Furthermore, whether increasing dosages would also result in even higher anti-GLA neutralizing antibody titers is unknown. Therefore, patients with severe disease progression who are running out of therapeutic options despite weight-adapted enzyme replacement therapy might also benefit from immune-modulating therapies. Transplant-related immunotherapy in patients with Fabry disease can significantly decrease the anti-GLA neutralizing antibody titers (Lenders et al., J Intern Med 282: 241-253 (2017)).

### IV. Kits

Also within the scope of the present disclosure are kits comprising the anti-GLA neutralizing antibody assay, as described herein, wherein the kit comprises:

(a) an assay buffer; (b) a substrate; (c) GALNAc inhibitor; (d) stop solution; and (e) an insert comprising instructions for use of the kit.

Kits typically include a label indicating the intended use of the contents of the kit and instructions for use. The term label includes any writing, or recorded material supplied on or with the kit, or which otherwise accompanies the kit.

All of the references cited above, as well as all references cited herein, are incorporated herein by reference in their entireties.

The following examples are offered by way of illustration and not by way of limitation

### EXAMPLES

### Example 1

### Anti-α-Galactosidase A (GLA) Neutralizing Antibody Assay

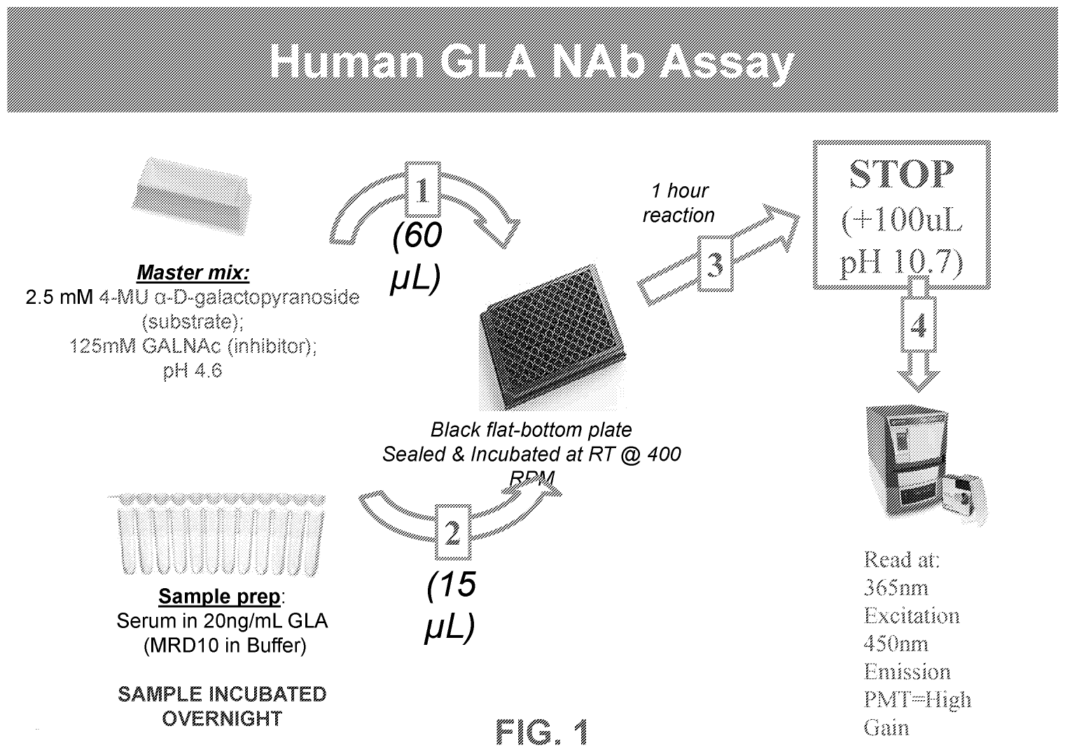

The presence of the anti-α-galactosidase A (GLA) neutralizing antibodies (NAbs) in serum was measured by high-throughput enzymatic assay for detecting NAbs in subjects with Fabry disease. The anti-GLA neutralizing antibody assay, as shown in FIG. 1, can be used to detect and monitor NAbs in Fabry patients and support therapeutics targeting Fabry disease. For example, it can be used in the clinic to semi-quantitatively determine the amount of neutralizing antibodies against α-galactosidase A, which can be prevalent for individuals with Fabry Disease (FD). The anti-GLA neutralizing antibody assay addresses the need for generating a suitable positive control, appropriately setting the cut-off point, overcoming and improving drug tolerance, and reducing the assay variability.

The assay utilized an artificial substrate, 4-methylumbelliferyl α-D-galactopyranoside (4-MU-α-Gal), to measure GLA activity and the inhibitory impact of neutralizing antibodies in human serum and a constant low level of GLA which is incubated with human serum overnight in an acidic assay buffer. After incubation, the sample was mixed with 4-MU-α-Gal substrate in a plate format. In the presence of catalytically active GLA, the substrate is cleaved and releases fluorescent 4-methylumbelliferone (4-MU) which can be quantitated at 365 nm excitation and 450n m emission at basic pHs.

The presence of neutralizing antibodies in the serum limits substrate cleavage and diminishes relative fluorescence unit (RFU) signal. Each sample was normalized against the fluorescence observed in negative wells to determine the relative % inhibition of the sample

$$( \% Inhibition = [ 1 - ( \frac{Sample RFU}{Mean NC RFU} ) ] \times 100 ) .$$

A sample is considered to be anti-GLA neutralizing antibody positive if a sample inhibition (% inhibition of α-Galactosidase A activity) is greater than the calculated cut-off threshold.

The human GLA protein was diluted to 40 ng/mL in an acidic sample buffer (mimicking lysosomal-like conditions) containing 0.1M citric acid, 0.2M sodium phosphate, and 0.05% Triton X-100 at pH 4.6. Human serum samples were diluted to 20% serum in a sample buffer and then mixed 1:1 with the diluted GLA buffer. The final sample incubation concentration contained 20 ng/mL GLA and 10% serum. Positive controls were prepared by utilizing human sera with known concentration of anti-GLA neutralizing antibodies (e.g., RP-01), while negative controls utilize human sera without the anti-GLA antibodies. The sample mixture is added in duplicate to a non-binding 96-well plate, sealed, and incubated overnight at 2-8° C.

After incubation, samples were transferred and diluted five-fold into a separate 96 well plate with reaction buffer containing: 2.5 mM 4-MU-α-Gal, 0.125 M GALNAc, 0.1M citric acid, 0.2M sodium phosphate and 0.05% Triton X-100 at pH 4.6. This reaction buffer continued to mimic lysosomal-like conditions while also containing 4-MU-α-Gal substrate, as well as GALNAc, an inhibitor of non-specific beta-galactosidase activity on the substrate. The reaction occurred for one hour at room temperature. The reaction was terminated with addition of a basic glycine solution containing 0.25M glycine at pH 10.7. The plate was then analyzed in a spectrophotometer at 365 nm wavelength excitation and 450 nm wavelength emission. All samples were normalized and evaluated against negative control wells containing negative sera.

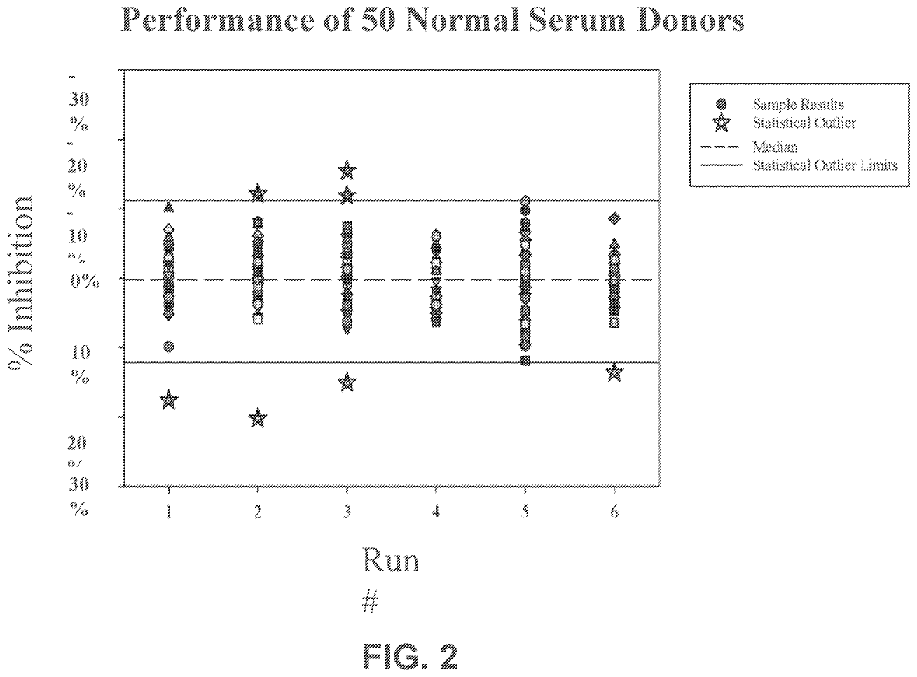

FIG. 2 shows performance of the assay across 6 different runs with 50 different serum donors. The assay exhibits low variability and high reproducibility, which allows a much lower positive threshold to be assigned for the presence of neutralizing antibodies than typical methods. A low threshold (cut-off, cut point) of about 10% increases the sensitivity of the assay.

### Example 2

### GAL Neutralizing Positive Control Antibody Screening and Assessment

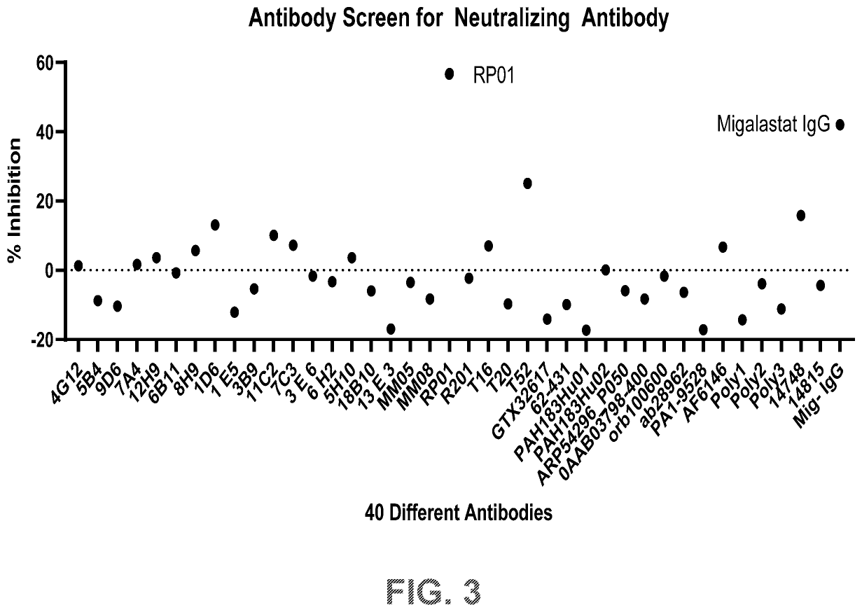

A neutralizing antibody screen of 40 different sources of anti-GLA antibodies was performed. The antibodies were tested for their enzymatic neutralizing ability (% inhibition of α-Galactosidase A activity) measured by the anti-GLA neutralizing antibody assay as described in Example 1. It is generally difficult to find an antibody that is capable of significant GLA enzyme inhibition. FIG. 3 shows that RP-01 was determined to be an effective positive control.

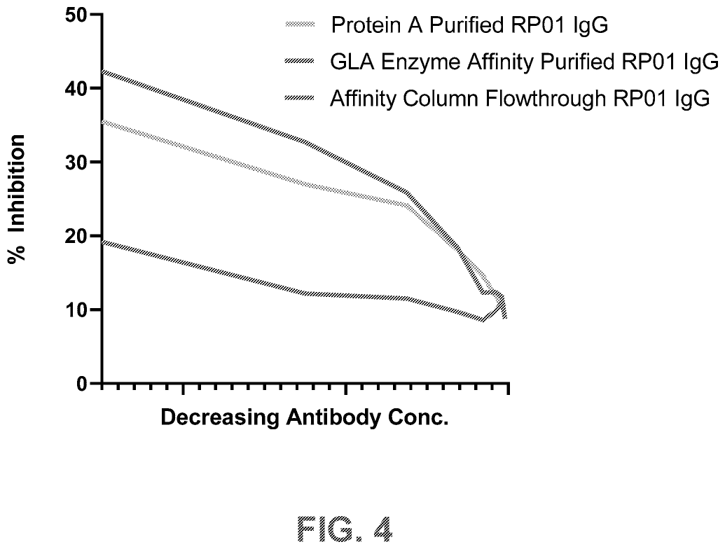

FIG. 4 shows the GLA neutralizing (positive control) antibody performance. RP-01 is a protein A purified polyclonal rabbit antibody, representing all circulating IgG from an immunized rabbit. RP-01 was affinity purified with traditional methods via an affinity column with cross-linked GLA. Affinity purified RP-01 is the antibody that was eluted and purified, while flow through RP-01 is the antibody that passed through the column. The flow through RP-01 contained all neutralizing antibodies, suggesting that traditional affinity purification methods for GLA may not purify neutralizing antibodies.

### Example 3

### Anti-α-Galactosidase A (GLA) Neutralizing Antibody Assay Optimization and Results

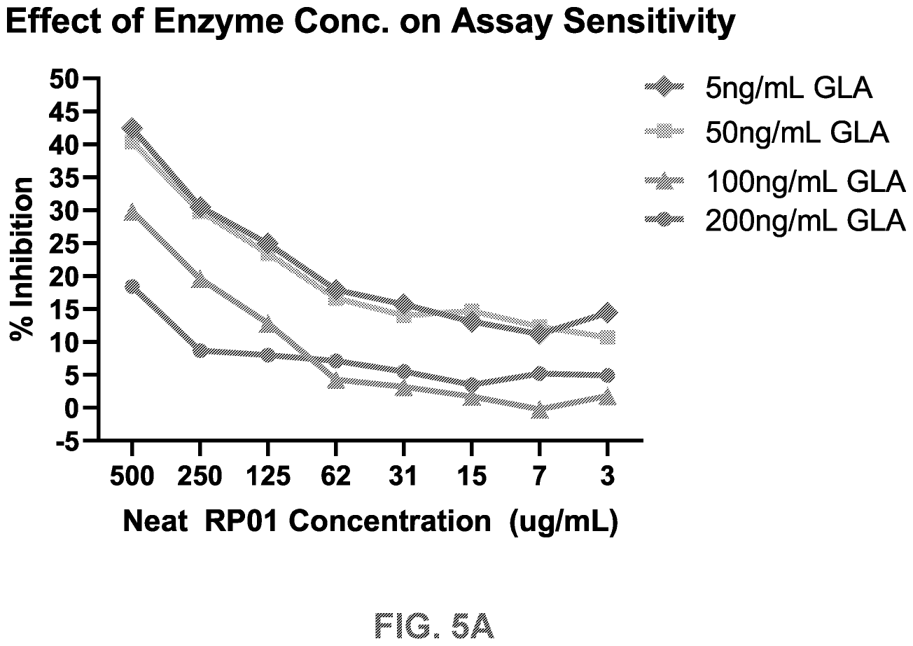

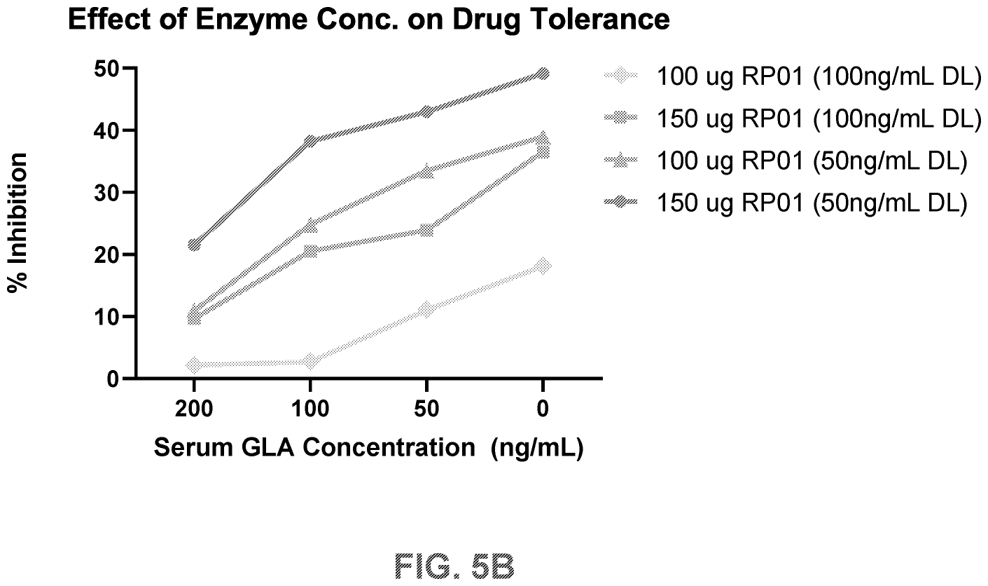

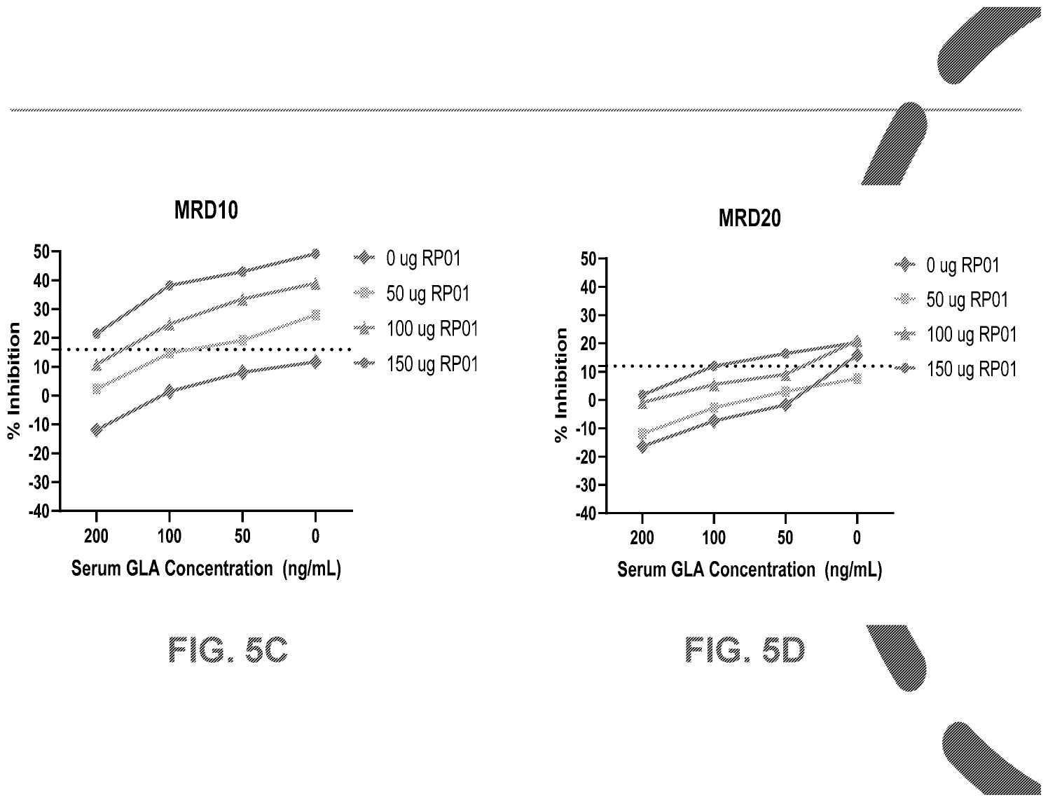

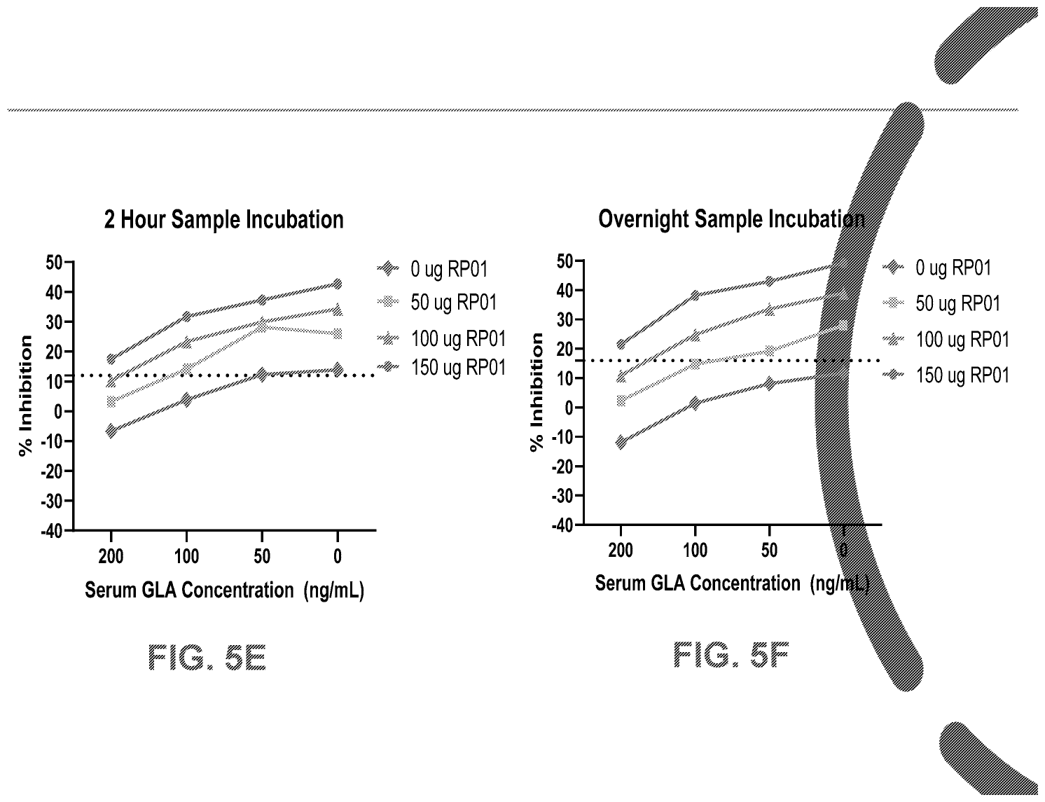

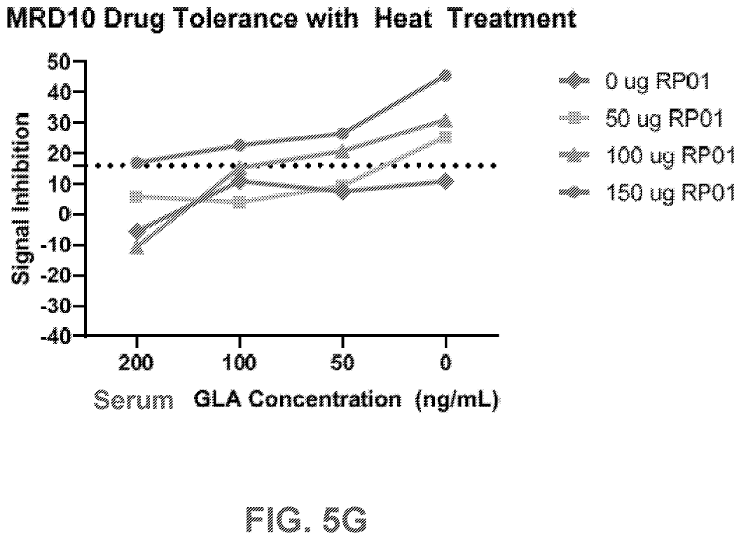

The anti-GLA neutralizing assay was optimized for sensitivity and drug tolerance by testing the enzymatic neutralizing ability (% inhibition of α-Galactosidase A activity) of RP-01 positive control antibody (as described in Example 1) under various assay conditions. FIG. 5A shows that the use of a lower concentration enzyme (GLA) in the assay buffer increases the enzymatic inhibition of GLA by RP-01. Use of a lower enzyme concentration in the assay provides additional sensitivity in comparison to typical methods.

FIG. 5B shows that the anti-GLA neutralizing antibody assay, as described herein, is tolerant to high levels of serum GLA. Use of low enzyme concentrations maintains high sensitivity without dramatically affecting the drug tolerance.

FIG. 5C shows that an initial dilution of human sera at 1:10 (MRD10) in assay buffer, increases the sensitivity of the assay in comparison to higher dilutions such as 1:20 (MRD20), as shown in FIG. 5D. Typical methods employ dilutions of at least 1:20 or greater.

Increasing the incubation time of the serum and GLA enzyme buffer from two hour sample incubation (FIG. 5E) to an overnight sample incubation (FIG. 5F) increases the inhibitory capacity of neutralizing antibodies, providing additional sensitivity. Typical methods employ limited incubation, if any.

Sample 56° C. pre-treatment eliminates free-GLA. FIG. 5G shows that the heat pre-treatment has limited effect in decreasing serum GLA when RP-01 antibody is present (i.e., IgG bound GLA).

Table 1 shows the signal to noise ratio (SNR) of the RP-01 titrations across 17 runs indicating high reproducibility of the RP-01 antibody at various concentrations (150 ug/ml, 75 ug/ml, 37.5 ug/ml, 18.75 ug/ml, 9.38 ug/ml, 4.69 ug/ml, 2.34 ug/ml, 1.17 ug/ml, 0.568 ug/ml, 0.293 ug/ml, and 0.146 ug/ml).

TABLE 1
Reproducibility of the RP-01 Antibody at Various Concentrations

| | Concentration (μg/ml) | | | | | | | | | | | |
|---|---|---|---|---|---|---|---|---|---|---|---|---|
| | 150 | 75.0 | 37.5 | 18.75 | 9.38 | 4.69 | 2.34 | 1.17 | 0.586 | 0.293 | 0.146 | |
| | Dilution Factor | | | | | | | | | | | |
| | Dil 1 | Dil 2 | Dil 4 | Dil 8 | Dil 16 | Dil 32 | Dil 64 | Dil 128 | Dil 256 | Dil 512 | Dil 1024 | Titer |
| Run ID | SNR | SNR | SNR | SNR | SNR | SNR | SNR | SNR | SNR | SNR | SNR | Result |
| 7RMRU2 | 0.599 | 0.700 | 0.815* | 0.925 | 0 962 | 0.997 | 1.010 | 1.030 | 1.080 | 1.050 | 1.050 | 4 |
| 8RMRU2 | 0.626 | 0.670 | 0.870* | 0.937 | 0.999 | 0.981 | 1.000 | 0.979 | 1.030 | 1.010 | 0.972 | 4 |
| 9RMRU2 | 0.621 | 0.716 | 0.829 | 0.893* | 0.980 | 0.974 | 1.010 | 1.020 | 1.060 | 1.050 | 1.030 | 8 |
| 10RMRU2 | 0.632 | 0.726 | 0.848* | 0.930 | 0.999 | 0.973 | 0.996 | 1.000 | 0.992 | 1.030 | 1.010 | 4 |
| 11RMRU2 | 0.595 | 0.704 | 0.858* | 0.936 | 0.974 | 0.980 | 0.984 | 0.987 | 0.977 | 1.020 | 0.980 | 4 |
| 12RMRU2 | 0.646 | 0.749 | 0.882 | 0.903* | 0.981 | 1.000 | 1.020 | 1.060 | 1.110 | 1.000 | 0.991 | 8 |
| 13RMRU2 | 0.626 | 0.676 | 0.808 | 0.869* | 0.924 | 0.949 | 0.936 | 0.940 | 1.010 | 0.982 | 1.040 | 8 |
| 14RMRU2 | 0.630 | 0.646 | 0.814 | 0.874* | 0.922 | 1.040 | 1.080 | 0.960 | 1.060 | 0.980 | 0.965 | 8 |
| 15RMRU2 | 0.604 | 0.690 | 0.825* | 0.914 | 0.963 | 0.976 | 0.997 | 1.000 | 1.030 | 1.030 | 1.020 | 4 |
| 16RMRU2 | 0.617 | 0.746 | 0.873* | 0.911 | 0 921 | 0.987 | 0.959 | 0.996 | 1.070 | 0.974 | 0.948 | 4 |
| 18RMRU2 | 0.591 | 0.665 | 0.862 | 0.904* | 0.953 | 0.964 | 0.938 | 1.019 | 1.100 | 1.070 | 1.060 | 8 |
| 19PMRU2 | 0.565 | 0.633 | 0.772 | 0.854* | 0.915 | 0.908 | 0.920 | 0.926 | 1.050 | 0.977 | 0.948 | 8 |
| 20PMRU2 | 0.559 | 0.645 | 0.778 | 0.860* | 0.923 | 0.879 | 0.903 | 0.970 | 1.030 | 1.020 | 0.981 | 8 |
| 21RMRU2 | 0.609 | 0.667 | 0.792 | 0.892 | 0.858* | 0.948 | 0.966 | 0.939 | 1.080 | 1.030 | 0.970 | 16 |
| 22RMRU2 | 0.614 | 0.702 | 0.798 | 0.884* | 0.958 | 0.947 | 0.983 | 0.944 | 1.000 | 0.939 | 0.958 | 8 |
| 23RMRU2 | 0.636 | 0.681 | 0.784 | 0.887* | 0.948 | 0.924 | 0.976 | 0.911 | 0.967 | 0.973 | 0.953 | 8 |
| 24RMRU2 | 0.624 | 0.715 | 0.792* | 0.911 | 0.969 | 0.944 | 0.974 | 0.972 | 0.956 | 0.997 | 0.991 | 4 |
| N | 17 | 17 | 17 | 17 | 17 | 17 | 17 | 17 | 17 | 17 | 17 | |
| Mean | 0.611 | 0.690 | 0.823 | 0.899 | 0.95 | 0.963 | 0.980 | 0.979 | 1.03 | 1.01 | 0.991 | |
| S.D. | 0.0239 | 0.0342 | 0.0359 | 0.0256 | 0.0361 | 0.0380 | 0.0418 | 0.0395 | 0.0459 | 0.0348 | 0.0355 | |
| % C.V. | 3.90 | 4.96 | 4.36 | 2.85 | 3.80 | 3.95 | 4.27 | 4.04 | 4.43 | 3.45 | 3.58 | |

Table 2 shows the GLA neutralizing antibody assay results (NAb results; % inhibition of α-Galactosidase A activity as described herein) tested in Fabry serum samples of three Fabry disease positive donors (Sample 1, Sample 2, and Sample 3). The anti-GLA neutralizing assay can detect relevant neutralizing antibodies in Fabry disease positive donors.

TABLE 2
Fabry Serum Sample GLA Neutralizing
Antibody (NAb) Assay Results

| | | NAb Results |
|---|---|---|
| | Sample | % inhibition |
| Fabry Serum Samples | Sample 1 | 82.5 |
| | Sample 2 | 94.3 |
| | Sample 3 | 57.7 |

What is claimed is:

1. A method of treating Fabry disease in a human subject in need thereof comprising administering a therapy for Fabry disease to the subject, wherein the subject is identified as an anti-α-Galactosidase A (GLA) neutralizing antibody negative subject when a biological sample of the subject is analyzed, wherein the anti-GLA neutralizing antibody negative subject has a biological sample having lower than about 30% inhibition of α-Galactosidase A activity as measured by an anti-GLA neutralizing antibody assay.

2. A method of identifying a human subject suitable for a therapy for Fabry disease comprising measuring the presence of an anti-GLA neutralizing antibody in a biological sample of the subject, wherein the subject suitable for a therapy is an anti-GLA neutralizing antibody negative subject when a biological sample of the subject is analyzed, wherein the anti-GLA neutralizing antibody negative subject has a biological sample having lower than about 30% inhibition of α-Galactosidase A activity as measured by an anti-GLA neutralizing antibody assay.

3. A method of treating Fabry disease in a human subject in need thereof comprising administering a therapy for Fabry disease to the subject, wherein the subject is identified as an anti-α-Galactosidase A (GLA) neutralizing antibody negative subject when a biological sample of the subject is analyzed, wherein biological sample is a serum sample, which is diluted at minimum required dilution (MRD)15 or lower.

4. A method of identifying a human subject suitable for a therapy for Fabry disease comprising measuring the presence of an anti-GLA neutralizing antibody in a biological sample of the subject, wherein the subject suitable for a therapy is an anti-GLA neutralizing antibody negative subject when a biological sample of the subject is analyzed, wherein biological sample is a serum sample, which is diluted at MRD15 or lower.

5. The method of claim 3 or 4, wherein the anti-GLA neutralizing antibody negative subject has a biological sample having lower than about 30% inhibition of α-Galactosidase A activity as measured by an anti-GLA neutralizing antibody assay.

6. The method of any one of claims 3 to 5, wherein the serum sample is mixed with α-Galactosidase A (GLA).

7. The method of claim 6, wherein the GLA is at a concentration of less than about 100 ng/ml, less than about 90 ng/ml, less than about 80 ng/ml, less than about 70 ng/ml, less than about 60 ng/ml, less than about 50 ng/ml, less than about 40 ng/ml, less than about 30 ng/ml, less than about ng/ml, or less than about 10 ng/ml.

8. The method of claim 7, wherein the GLA is at a concentration of about 20 ng/ml.

9. The method of any one of claims 6 to 8, wherein the serum sample and the GLA mixture are incubated for at least about one hour, at least about two hours, at least about three hours, at least about four hours, at least about five hours, at least about six hours, at least about seven hours, at least about eight hours, at least about nine hours, at least about ten hours, at least about eleven hours, at least about twelve hours, at least about thirteen hours, at least about fourteen hours, at least about fifteen hours, at least about sixteen hours, at least about seventeen hours, at least about eighteen hours, at least about nineteen hours, at least about twenty hours, at least about twenty one hours, at least about twenty two hours, at least about twenty three hours, or at least about twenty four hours.

10. The method of any one of claims 6 to 8, wherein the serum sample and the GLA mixture are incubated for a duration between about 1 and about 15 hours, between about 2 and about 14 hours, between about 3 and about 13 hours, between about 4 and about 12 hours, between about 5 and about 11 hours, between about 6 and about 10 hours, or between about 7 and about 9 hours.

11. The method of any one of claims 3 to 10, wherein the serum sample is mixed with a reaction mix.

12. The method of claim 11, wherein the reaction mix comprises a substrate and an inhibitor.

13. The method of claim 12, wherein the substrate comprises 4-methylumbelliferyl (4-MU)-α-D-galactopyranoside or 4-Nitrophenyl α-D-galactopyranoside.

14. The method of claim 12 or 13, wherein the substrate is at a concentration of at least about 1.1 mM, at least about 1.2 mM, at least about 1.3 mM, at least about 1.4 mM, at least about 1.5 mM, at least about 1.6 mM, at least about 1.7 mM, at least about 1.8 mM, at least about 1.9 mM, at least about 2 mM, at least about 2.1 mM, at least about 2.2 mM, at least about 2.3 mM, at least about 2.4 mM, at least about 2.5 mM, at least about 2.6 mM, at least about 2.7 mM, at least about 2.8 mM, at least about 2.9 mM, at least about 3 mM, at least about 3.1 mM, at least about 3.2 mM, at least about 3.3 mM, at least about 3.4 mM, at least about 3.5 mM, at least about 3.6 mM, at least about 3.7 mM, at least about 3.8 mM, at least about 3.9 mM. at least about 4 mM, at least about 4.1 mM, at least about 4.2 mM, at least about 4.3 mM, at least about 4.4 mM, at least about 4.5 mM, at least about 4.6 mM, at least about 4.7 mM, at least about 4.8 mM, at least about 4.9 mM, or at least about 5 mM.

15. The method of claim 12, wherein the inhibitor comprises N-Acetylgalactosamine (GALNAc).

16. The method of claim 12 or 15, where the inhibitor is at a concentration of less than about 200 mM, less than about 195 mM, less than about 190 mM, less than about 185 mM, less than about 180 mM, less than about 175 mM, less than about 170 mM, less than about 165 mM, less than about 160 mM, less than about 155 mM, less than about 150 mM, less than about 145 mM, less than about 140 mM, less than about 135 mM, less than about 130 mM, less than about 125 mM, less than about 120 mM, less than about 115 mM, or less than about 110 mM.

17. The method of any one of claims 11 to 16, wherein the reaction mix and the serum sample are mixed in a high throughput plate.

18. The method of claim 17, wherein the reaction mix and the serum sample mixture are incubated at room temperature at revolutions per minute (RPM) 300, 400, 500, or 600.

19. The method of claim 18, further comprising adding a stop buffer to the mixture after incubation.

20. The method of claim 18 or 19, wherein the incubation period is at least about 30 minutes, at least about 35 minutes, at least about 40 minutes, at least about 45 minutes, at least about 50 minutes, at least about 55 minutes, at least about 60 minutes, at least about 65 minutes, at least about 70 minutes, at least about 75 minutes, or at least about 80 minutes.

21. The method of claim 19 or 20, wherein the stop buffer comprises glycine.

22. The method of any one of claims 19 to 21, wherein the stop buffer is at a volume of less than about 1 mL, less than about 900 uL, less than about 800 uL, less than about 700 uL, less than about 600 uL, less than about 500 uL, less than about 400 uL, less than about 300 uL, less than about 200 uL, or less than about 100 uL.

23. The method of any one of claims 2 and 4 to 22, further comprising administering Fabry disease therapy.

24. The method of any one of claims 1, 2, and 5 to 23, wherein the sample has about 29%, about 28%, about 27%, about 26%, about 25%, about 24%, about 23%, about 22%, about 21%, or about 20% inhibition of α-Galactosidase A activity.

25. The method of any one of claims 1, 2, and 5 to 23, wherein the sample has lower than about 15%, about 16%, about 17% about 18%, about 19%, about 20%, about 21%, about 22%, about 23%, about 24%, about 25%, about 26%, about 27%, about 28%, about 29%, or about 30% inhibition of α-Galactosidase A activity.

26. The method of any one of claims 1, 2, and 5 to 23, wherein the sample has lower than about 27% inhibition of α-Galactosidase A activity.

27. The method of any one of claims 1, 2, and 5 to 23, wherein the sample has lower than about 20% inhibition of α-Galactosidase A activity.

28. The method of any one of claims 1, 2, and 5 to 23, wherein the sample has lower than about 15% inhibition of α-Galactosidase A activity.

29. The method of any one of claims 1, 2, and 5 to 23, wherein the sample has lower than about 10% inhibition of α-Galactosidase A activity.

30. The method of any one of claims 1, 2, and 5 to 23, wherein the sample has lower than 1% inhibition of α-Galactosidase A activity.

31. The method of any one of claims 1, 2, and 5 to 23, wherein the sample has no inhibition of α-Galactosidase A activity.

32. The method of any one of claims 1, 2, and 5 to 23, wherein the sample has about 1% to about 10% inhibition of α-Galactosidase A activity.

33. The method of any one of claims 1, 2, and 5 to 23, wherein the sample has about 10% to about 20% inhibition of α-Galactosidase A activity.

34. The method of any one of claims 1, 2, and 5 to 23, wherein the sample has about 20% to about 30% inhibition of α-Galactosidase A activity.

35. The method of any one of claims 1 to 34, wherein the subject has been administered with an enzyme replacement therapy for Fabry disease prior to the administering and/or the measuring (“pre-treatment”).

36. The method of any one of claims 2 and 4 to 34, wherein the therapy for Fabry disease is an enzyme replacement therapy.

37. The method of claim 35 or 36, wherein the enzyme replacement therapy comprises a recombinant α-Galactosidase A (GLA) protein or a gene expressing GAL.

38. The method of claim 37, wherein the enzyme replacement therapy comprises administering galafold, ST-920, AVR-RD-01, FLT-190, or any combination thereof.

39. The method of any one of claims 35 to 37, wherein the enzyme replacement therapy comprises a recombinant α-Galactosidase A (GLA) protein in combination with an active site-specific chaperone (ASSC) for the GLA.

40. The method of claim 39, wherein the ASSC is 1-deoxygalactonojirimycin.

41. The method of claim 35 or 36, wherein the enzyme replacement therapy comprises agalsidase alpha and/or beta or a gene expressing agalsidase alpha and/or beta.

42. The method of claim 41, wherein the enzyme replacement therapy comprises fabrazyme, Replagal, PRX-102, or any combination thereof.

43. The method of claim 37 or 41, wherein the enzyme replacement therapy comprises a gene therapy.

44. The method of claim 43, wherein the gene therapy comprises a vector encoding the enzyme.

45. The method of claim 43, wherein the gene therapy comprises administering ST-920, AVR-RD-01, FLT-190, or any combination thereof.

46. The method of claim 44, wherein the vector comprises an mRNA encoding a human GLA protein or agalsidase alpha and/or beta.

47. The method of claim 44, wherein the vector is a viral vector.

48. The method of claim 47, wherein the viral vector comprises an adeno-associated virus (AAV) vector or a lentiviral vector.

49. The method of claim 43, wherein the gene therapy is delivered by a lipid nanoparticle.

50. The method of any one of claims 2 and 4 to 34, wherein the therapy for Fabry disease comprises a non-enzyme replacement therapy.

51. The method of claim 50, wherein the therapy for Fabry disease comprises lucerastat, venglustat, apabetalone, or any combination thereof.

52. The method of claim 35, wherein the pre-treatment is an enzyme replacement therapy.

53. The method of claim 52, wherein the enzyme replacement therapy for the pre-treatment comprises a recombinant α-Galactosidase A (GLA) protein or a gene expressing GAL.

54. The method of claim 53, wherein the enzyme replacement therapy for the pre-treatment comprises administering galafold, ST-920, AVR-RD-01, FLT-190, or any combination thereof.

55. The method of claim 53 or 54, wherein the enzyme replacement therapy for the pre-treatment comprises agalsidase alpha and/or beta or a gene expressing agalsidase alpha and/or beta.

56. The method of claim 55, wherein the enzyme replacement therapy for the pre-treatment comprises fabrazyme, Replagal, PRX-102, or any combination thereof.

57. The method of claim 53 or 55, wherein the enzyme replacement therapy for the pre-treatment comprises a gene therapy.

58. The method of claim 57, wherein the gene therapy comprises a vector encoding the enzyme.

59. The method of claim 57, wherein the gene therapy comprises administering ST-920, AVR-RD-01, FLT-190, or any combination thereof.

60. The method of claim 58, wherein the vector comprises an mRNA encoding a human GLA protein or agalsidase alpha and/or beta.

61. The method of claim 58, wherein the vector is a viral vector.

62. The method of claim 61, wherein the viral vector comprises an adeno-associated virus (AAV) vector or a lentiviral vector.

63. The method of claim 57, wherein the gene therapy is delivered by a lipid nanoparticle.

64. The method of any one of claims 1 to 63, wherein Fabry disease is type 1 classic phenotype or type 2 later-onset phenotype.

65. The method of claim 1 or 2, wherein the biological sample is serum.

66. The method of any one of claims 1, 2, 5, 6, and 9 to 65, wherein the anti-GLA neutralizing antibody assay is standardized by an antibody designated as RP-01.

67. The method of claim 66, wherein the assay standardization comprises determining a GLA drug diluent concentration by measuring the effect of the GLA drug level on the inhibitory effect of the RP-01 antibody.

68. A method of standardizing an anti-GLA neutralizing antibody assay comprising determining a GLA drug diluent concentration by measuring the effect of the GLA drug level on the inhibitory effect of an antibody designated as RP-01.

69. The method of claim 68, wherein the standardization comprises determining the GLA drug diluent concentration by measuring the effect of the GLA drug level on the inhibitory effect of the RP-01 antibody.

70. The method of any one of claims 67 to 69, wherein the GLA drug diluent concentration is less than about 100 ng/ml, less than about 90 ng/ml, less than about 80 ng/ml, less than about 70 ng/ml, less than about 60 ng/ml, less than about 50 ng/ml, less than about 40 ng/ml, less than about ng/ml, less than about 20 ng/ml, or less than about 10 ng/ml.

71. The method of claim 70, wherein the GLA drug diluent concentration is about 40 ng/ml.

72. The method of claim 70, wherein the GLA drug diluent concentration is about 20 ng/ml.

73. The method of any one of claims 67 to 72, wherein the inhibitory effect of the RP-01 antibody is represented as % inhibition of α-Galactosidase A activity as measured by the anti-GLA neutralizing antibody assay using different concentrations of the RP-01 antibody.

74. The method of claim 73, wherein the inhibitory effect of RP-01 is represented as about 20% inhibition of α-Galactosidase A activity at about 50 ug/ml RP-01 concentration.

75. The method of claim 73, wherein the inhibitory effect of RP-01 is represented as about 30% inhibition of α-Galactosidase A activity at about 100 ug/ml RP-01 concentration.

76. The method of claim 73, wherein the inhibitory effect of RP-01 is represented as about 40% inhibition of α-Galactosidase A activity at about 150 ug/ml RP-01 concentration.

77. The method of any one of claims 67 to 76, wherein the GLA drug has a minimal required dilution (MRD) of about 1 fold, about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, about 30 fold, about 31 fold, about 32 fold, about 33 fold, about 34 fold, about 35 fold, about 36 fold, about 37 fold, about 38 fold, about 39 fold, about 40 fold, about 41 fold, about 42 fold, about 43 fold, about 44 fold, about 45 fold, about 46 fold, about 47 fold, about 48 fold, about 49 fold, about 50 fold, about 51 fold, about 52 fold, about 53 fold, about 54 fold, about 55 fold, about 56 fold, about 57 fold, about 58 fold, about 59 fold, about 60 fold, about 61 fold, about 62 fold, about 63 fold, about 64 fold, about 65 fold, about 66 fold, about 67 fold, about 68 fold, about 69 fold, about 70 fold, about 71 fold, about 72 fold, about 73 fold, about 74 fold, about 75 fold, about 76 fold, about 77 fold, about 78 fold, about 79 fold, about 80 fold, about 81 fold, about 82 fold, about 83 fold, about 84 fold, about 85 fold, about 86 fold, about 87 fold, about 88 fold, about 89 fold, about 90 fold, about 91 fold, about 92 fold, about 93 fold, about 94 fold, about 95 fold, about 96 fold, about 97 fold, about 98 fold, about 99 fold, or about 100 fold.

78. A kit comprising the anti-GLA neutralizing antibody assay of any one of claims 1 to 77, wherein the kit comprises:
(a) an assay buffer
(b) a substrate
(c) GALNAc inhibitor
(d) stop solution; and
(e) an insert comprising instructions for use of the kit.

79. A method of treating Fabry disease in a human subject in need thereof comprising administering a non-enzyme replacement therapy for Fabry disease to the subject, wherein the subject is identified as an anti-α-Galactosidase A (GLA) neutralizing antibody positive subject when a biological sample of the subject is analyzed, wherein the anti-GLA neutralizing antibody positive subject has a biological sample having higher than about 10% inhibition of α-Galactosidase A activity as measured by an anti-GLA neutralizing antibody assay.

80. A method of identifying a human subject suitable for a non-enzyme replacement therapy for Fabry disease comprising measuring the presence of an anti-GLA neutralizing antibody in a biological sample of the subject, wherein the subject suitable for a therapy is an anti-GLA neutralizing antibody positive subject when a biological sample of the subject is analyzed, wherein the anti-GLA neutralizing antibody positive subject has a biological sample having higher than about 10% inhibition of α-Galactosidase A activity as measured by an anti-GLA neutralizing antibody assay.

81. The method of claim 80, further comprising administering a therapy that is not an enzyme replacement therapy.

82. A method of identifying a human subject who is not eligible for an enzyme replacement therapy for Fabry disease, comprising measuring the presence of an anti-GLA neutralizing antibody in a biological sample of the subject, wherein the sample having higher than about 10% inhibition of α-Galactosidase A activity as measured by the anti-GLA neutralizing antibody assay is identified as the anti-GLA neutralizing antibody positive sample, and wherein the subject not eligible for the enzyme replacement therapy for Fabry disease has the anti-GLA neutralizing antibody positive sample.

83. The method of claim 82, further comprising administering Fabry disease therapy that is not an enzyme replacement therapy.

84. The method of any one of claims 80, 81, and 83, wherein the therapy comprises administering lucerastat, venglustat, or apabetalone.

85. The method of any one of claims 66 to 69 and 73 to 76, wherein the RP-01 is a polyclonal antibody.
